SEQUENCE LISTING (4 sequences)

SEQ ID NO: 1 - DNA, 59 bp, Artificial Sequence
  synthetic
tcagatacatagatacaattctattacccccatccatacagacatggaggcccagaata

SEQ ID NO: 2 - DNA, 60 bp, Artificial Sequence
  synthetic
aagtaggtttatacataattttacaactcattacgcacaccagtatagcgaccagcattc

SEQ ID NO: 3 - DNA, 22 bp, Artificial Sequence
  synthetic
ggttggcaaatgactaattaag

SEQ ID NO: 4 - DNA, 20 bp, Artificial Sequence
  synthetic
cagtatagcgaccagcattc

(19) United States
(12) Patent Application Publication
(10) Pub. No.: US 2023/0227860 A1
(43) Pub. Date: Jul. 20, 2023

(54) INORGANIC-BIOLOGICAL HYBRID SYSTEM FOR BIOFUEL PRODUCTION

(71) Applicant: Massachusetts Institute of Technology, Cambrige, MA (US)

(72) Inventors: Shalmalee Pandit, Cambridge, MA (US); Angela Belcher, Lexington, MA (US)

(73) Assignee: Massachusetts Institute of Technology, Cambridge, MA (US)

(21) Appl. No.: 18/001,032

(22) PCT Filed: Jun. 9, 2021
(86) PCT No.: PCT/US2021/036624

§ 371 Date: Dec. 7, 2022

Related U.S. Application Data
(60) Provisional application No. 63/037,546, filed on Jun. 10, 2020.

(51) Int. Cl. C12P 7/06; C12N 13/00

(52) U.S. Cl. CPC C12P 7/06; C12N 13/00; B82Y 5/00

(57) ABSTRACT

A system for biofuel production can include a cell, a nanoparticle on a surface of the cell, and an irradiation unit configured to expose the cell to irradiation. A method of producing biofuel can include providing a cell having a nanoparticle on a surface of the cell, exposing the cell to a fuel precursor, irradiating the cell, converting the fuel precursor to a biofuel with the cell, and collecting the biofuel.

### PRIORITY CLAIM

This application claims priority to U.S. Provisional Application No. 63/037,546, filed Jun. 10, 2021, which is incorporated by reference in its entirety.

### STATEMENT REGARDING FEDERALLY SPONSORED RESEARCH OR DEVELOPMENT

This invention was made with Government support under Grant No. HR0011-18-2-0049 awarded by the Defense Advanced Research Projects Agency (DARPA). The Government has certain rights in the invention.

### SEQUENCE LISTING

A Sequence Listing accompanies this application and is submitted as an ASCII text file of the sequence listing named “MIT_21974_ST25.txt” which is 1 KB in size and was created on Jun. 8, 2021. The sequence listing is electronically submitted via EFS-Web with the application and is incorporated herein by reference in its entirety.

### TECHNICAL FIELD

This invention relates to biofuels.

### BACKGROUND

Factors such as economic security, environmental protection, and sustainability of resources have driven research pertaining to the production of fuels from renewable resources. See, Eagan, N. M., Kumbhalkar, M. D., Buchanan, J. S., Dumesic, J. A. & Huber, G. W. Chemistries and processes for the conversion of ethanol into middle-distillate fuels. Nat. Rev. Chem. 3, 223-249 (2019), and Dehghani Madvar, M., Aslani, A., Ahmadi, M. H. & Karbalaie Ghomi, N. S. Current status and future forecasting of biofuels technology development. Int. J. Energy Res. 43, 1142-1160 (2019), each of which is incorporated by reference in its entirety. While other sources of renewable energy are useful for electrical power, residential, or commercial purposes, liquid fuels are required for use of the transportation sector. The global demand for a renewable fuel for the transportation sector is expected to increase over the next two decades. See, Outlook for Energy: A perspective to 2040 | ExxonMobil, available at: corporate.exxonmobil.com/Energy-and-environment/Looking-forward/Outlook-for-Energy/Outlook-for-Energy-A-perspective-to-2040 (accessed: 9th January 2020), which is incorporated by reference in its entirety.

### SUMMARY

In one aspect, a system for production of a chemical product can include a cell, a nanoparticle on a surface of the cell, and an irradiation unit configured to expose the cell to irradiation.

In another aspect, a method of producing a chemical product can include providing a cell having a nanoparticle on a surface of the cell, exposing the cell to a precursor, irradiating the cell, converting the precursor to a chemical product with the cell, and collecting the chemical product. In certain circumstances, irradiating can include irradiating ultraviolet (UV) light.

In certain circumstances, the chemical product can be a biofuel, for example, ethanol. In certain circumstances, the precursor can include glucose or carbon dioxide.

In certain circumstances, the cell can be a yeast cell.

In certain circumstances, a thiol synthesis pathway can be deleted from the cell. In certain circumstances, the thiol synthesis pathway can include Met17.

In certain circumstances, the nanoparticle can include cadmium. For example, the nanoparticle can include cadmium sulfide.

In certain circumstances, the irradiation unit can include an ultraviolet (UV) light source.

In certain circumstances, the system can include a bioreactor including the irradiation unit configured to irradiate contents of the bioreactor.

Other aspects, embodiments, and features will be apparent from the following description, the drawings, and the claims.

### BRIEF DESCRIPTION OF THE DRAWINGS

FIGS. 1A-1F show yeast-nanoparticle hybrid system. FIGS. 1 shows schematic of yeast-hybrid system. FIG. 1B shows a schematic of treating the ΔMet17 strain with cadmium ions (Cd²⁺) leads to formation of CdS nanoparticles on the cell surface. FIG. 1C shows a TEM image of ΔMet17 cross-section to displaying the localization of CdS nanoparticles on the cell surface. FIG. 1D shows elemental mapping analysis of the TEM image measuring and presenting the location of cadmium in the sample. FIG. 1E shows elemental mapping analysis of the TEM image measuring and presenting the location of sulfur in the sample. FIG. 1F shows elemental mapping analysis measuring and displaying the location of both cadmium (red) and sulfur (blue) in the sample.

FIGS. 2A-2E show analysis of the transcriptomic changes due to ΔMet17 mutation, cadmium ion, and light treatment through RNA Sequencing. FIG. 2A shows principal Component Analysis of RNA Sequencing data. FIG. 2B shows log two fold change volcano plot depicting the effects of the precipitation of cadmium sulfide nanoparticles on the yeast transcriptome. FIG. 2C shows log two fold change volcano plot illustrating the effects of light treatment on the yeast transcriptome. FIG. 2D shows gene set enrichment analysis displaying the effects of light on protein coding genes. FIG. 2E shows gene set enrichment analysis characterizing the effects of cadmium sulfide nanoparticles on protein coding genes.

FIGS. 3A-3C show metabolomic characterization of yeast strains with varied treatment conditions. FIG. 3A shows NAD⁺ to NADH ratio in W303α and ΔMet17 yeast strains. FIG. 3B shows ATP to ADP ratio in W303α and ΔMet17 yeast strains. FIG. 3C shows LC-MS data analyzing nutrients in the supernatant media displayed with intracellular ethanol concentration in yeast strains with treatment conditions.

FIGS. 4A-4F show deletion of Met17 in another yeast strain and characterizing the effects. FIG. 4A shows a TEM image of Y567 ΔMet17 strain. FIG. 4B shows elemental mapping analysis of the cell surface of Y567 ΔMet17 displays the localization of cadmium in the sample. FIG. 4C shows elemental mapping analysis of the TEM image measures and presents the location of sulfur in the sample. FIG. 4D shows elemental mapping analysis measures and displays the location of both cadmium (red) and sulfur (blue) in the sample. FIG. 4E shows NAD⁺ to NADH ratio in Y567 and Y567 ΔMet17 yeast strains. FIG. 4F shows ATP to ADP ratio in Y567 and Y567 ΔMet17 yeast strains.

FIGS. 5A-5F show ethanol production through yeast-nanoparticle hybrid system. FIG. 5A shows intracellular ethanol concentration in W303α and ΔMet17 yeast strains. FIG. 5B shows intracellular ethanol concentration in Y567 and Y567 ΔMet17 yeast strains. FIG. 5C shows concentration of ethanol in the supernatant media of W303α and ΔMet17 yeast strains. FIG. 5D shows concentration of ethanol in the supernatant media of Y567 and Y567 ΔMet17 yeast strains. FIG. 5E shows concentration of glucose in the supernatant media of W303α and ΔMet17 yeast strains. FIG. 5F shows concentration of glucose in the supernatant media of Y567 and Y567 ΔMet17 yeast strains.

FIGS. 6A-6D show ΔMet17 treated with Cd²⁺.FIG. 6A shows a TEM image of ΔMet17 treated with Cd²⁺. FIG. 6B shows elemental mapping analysis measuring cadmium. FIG. 6C shows elemental mapping analysis measuring sulfur. FIG. 6D shows elemental mapping analysis measuring both cadmium (red) and sulfur (blue).

FIGS. 7A-7B show W303α treated with Cd²⁺. FIG. 7A shows a TEM image of W303α yeast treated with Cd²⁺. FIG. 7B shows elemental mapping analysis measuring both cadmium (red) and sulfur (blue) simultaneously.

FIG. 8 shows a TEM image of W303α treated with cadmium ions (Cd²⁺).

FIG. 9 shows experimental conditions for light/dark experiments.

FIG. 10 shows a heatmap of RNA sequencing data clustered by similarity in gene expression.

FIG. 11 shows total NAD+ and NADH in yeast strains with various treatment conditions.

FIG. 12 shows total ATP and ADP in yeast strains with various treatment conditions. Supplementary

FIG. 13 shows absolute values of excretion and consumption rates through liquid chromatography-mass spectrometry analysis.

FIGS. 14A-14B show Y567 treated with Cd²⁺. FIG. 14A shows a TEM image of Y567 yeast treated with Cd²⁺. FIG. 14B shows elemental mapping analysis measuring both cadmium (red) and sulfur (blue) simultaneously

FIG. 15 shows emission at maximum excitation of 350 nm of CdS nanoparticles extracted from Y567 ΔMet17.

FIG. 16 shows carbon dioxide fixation.

FIG. 17 shows a schematic of a bioreactor.

FIGS. 18A-18G show labeling results.

### DETAILED DESCRIPTION

Artificially photosynthetic systems can aim to store solar energy and chemically reduce carbon dioxide. These systems can use light to drive processes for carbon fixation into biomass and/or liquid fuels. In particular, a system including a cell decorated with semiconductor nanoparticles that is irradiated can produce a product with a higher yield than without the irradiation.

For example, engineered photosynthetic systems aim to capture solar energy and reduce carbon dioxide. These systems use light to create conditions favorable for net carbon fixation to produce biomass and/or liquid fuels. A hybrid inorganic-biological system is described that combines the light harvesting properties of a semiconductor system that when combined with genetic engineering can alter yeast cell redox state and favor generation of useful products. Here it is shown that this system can be used to increase ethanol production, a common biofuel, through reductive carboxylation stimulated by biologically produced cadmium sulfide nanoparticles and light. This illustrates how use of this system can alter yeast metabolism and allow production of many metabolites.

In general, a system has been developed that harvests light and drives an oxidized cell state. The altered metabolic state favors the system’s increased ability to fix carbon and produce biofuel.

Disclosed herein is a hybrid inorganic-biological system that can utilize an input of toxic waste to drive product formation. In one aspect, the hybrid system can produce a chemical product, such as biomass or a biofuel. In certain embodiments, the biofuel can be ethanol. In certain embodiments, the inorganic system can include nanoparticles. In certain embodiments, the biological system can include cells. For example, a system for production of a chemical product can include a cell, a nanoparticle on a surface of the cell, and an irradiation unit configured to expose the cell to irradiation. A method of production of a chemical product can include providing a cell having a nanoparticle on a surface of the cell, exposing the cell to a fuel precursor, irradiating the cell, converting the precursor to a chemical product with the cell, and collecting the chemical product. In certain embodiments, the cell can be yeast cell. For example, the system endogenously can generate nanoparticles that through light stimulus, activate the yeast. In certain embodiments, the yeast can produce an increased amount of a biofuel, such as ethanol when irradiated compared to when not irradiated.

The hybrid inorganic-biological system can manage both genetically controlled generation of products along with the ability to photoactivate a semiconductor system. For example, an increase in the production of a chemical product such as ethanol, a common biofuel, through the electron transfer can be stimulated by biologically produced nanoparticles and light. In certain embodiments, the nanoparticles can include cadmium. In certain embodiments, nanoparticles can include cadmium sulfide. This system can improve the production of many metabolites and products through endogenously produced nanoparticles.

In one aspect, a system for production of a chemical product can include a cell, a nanoparticle on a surface of the cell, and an irradiation unit configured to expose the cell to irradiation. For example, a method of producing a chemical product can include providing a cell having a nanoparticle on a surface of the cell, exposing the cell to a precursor, irradiating the cell, converting the precursor to a chemical product with the cell, and collecting the chemical product. In certain circumstances, irradiating can include irradiating ultraviolet (UV) light.

The chemical product can form by transformation of a precursor, which can be a biologically-available substrate. For example, the precursor can include glucose or carbon dioxide The chemical product can be an organic molecule or other target, such as a biofuel. For example, the chemical product can be ethanol.

In certain circumstances, the cell can be a yeast cell. For example, the cell can be a transformed cell as described, for example, in PCT/US2018/016576, which is incorporated by reference in its entirety. For example, a thiol synthesis pathway can be deleted from the cell. In certain circumstances, the thiol synthesis pathway can include Met17. Cells with this modification can present a nanoparticle on the surface of a cell.

In certain circumstances, the nanoparticle can include cadmium. For example, the nanoparticle can include cadmium sulfide.

The nanoparticle can be a nanocrystal. In certain circumstances, the nanoparticle can include a semiconductor material. The semiconductor material forming the nanoparticle can include a Group II-VI compound, a Group II-V compound, a Group III-VI compound, a Group III-V compound, a Group IV-VI compound, a Group I-III-VI compound, a Group II-IV-VI compound, or a Group II-IV-V compound, for example, ZnO, ZnS, ZnSe, ZnTe, CdO, CdS, CdSe, CdTe, MgO, MgS, MgSe, MgTe, HgO, HgS, HgSe, HgTe, AlN, AlP, AlAs, AlSb, GaN, GaP, GaAs, GaSb, InN, InP, InAs, InSb, TlN, TlP, TlAs, TlSb, TlSb, PbS, PbSe, PbTe, Cd₃As₂, Cd₃P₂ or mixtures thereof.

In certain circumstances, the irradiation unit can include an ultraviolet (UV) light source. The nanoparticle can be irradiated with a wavelength of light, for example, the nanoparticle can be excited with light having a wavelength of 500 nm or shorter, 450 nm or shorter, 400 nm or shorter, or 350 nm or shorter.

The nanoparticles can be formed by exposing the cell to an M-containing salt. Suitable M-containing salts include cadmium acetylacetonate, cadmium iodide, cadmium bromide, cadmium chloride, cadmium hydroxide, cadmium carbonate, cadmium acetate, cadmium myristate, cadmium oleate, cadmium oxide, zinc acetylacetonate, zinc iodide, zinc bromide, zinc chloride, zinc hydroxide, zinc carbonate, zinc acetate, zinc myristate, zinc oleate, zinc oxide, magnesium acetylacetonate, magnesium iodide, magnesium bromide, magnesium chloride, magnesium hydroxide, magnesium carbonate, magnesium acetate, magnesium myristate, magnesium oleate, magnesium oxide, mercury acetylacetonate, mercury iodide, mercury bromide, mercury chloride, mercury hydroxide, mercury carbonate, mercury acetate, mercury myristate, mercury oleate, aluminum acetylacetonate, aluminum iodide, aluminum bromide, aluminum chloride, aluminum hydroxide, aluminum carbonate, aluminum acetate, aluminum myristate, aluminum oleate, gallium acetylacetonate, gallium iodide, gallium bromide, gallium chloride, gallium hydroxide, gallium carbonate, gallium acetate, gallium myristate, gallium oleate, indium acetylacetonate, indium iodide, indium bromide, indium chloride, indium hydroxide, indium carbonate, indium acetate, indium myristate, indium oleate, thallium acetylacetonate, thallium iodide, thallium bromide, thallium chloride, thallium hydroxide, thallium carbonate, thallium acetate, thallium myristate, or thallium oleate.

The nanoparticle can have a size of less than 150 Å, for example, average diameters in the range of 10 Å to 125 Å.

The cell can be mutated to be sensitive for a metal, which can lead to nanoparticle formation. For example, the cells can be were screened by subjecting libraries to 100 µM metal ions in culture and fractionated based on density changes. See, for example, PCT/US2018/016576, which is incorporated by reference in its entirety. The cell can be decorated with the nanoparticle by exposing the cell to the M-contained salt.

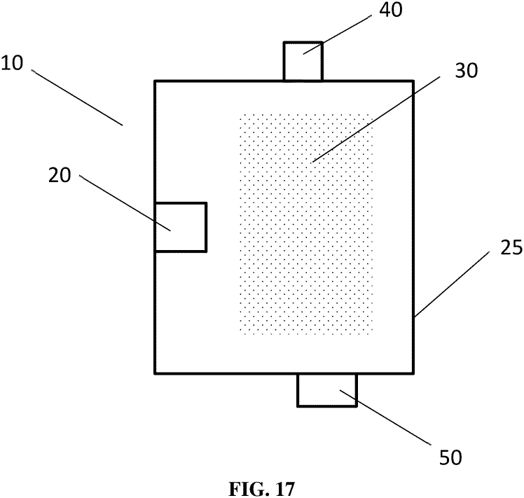

In certain circumstances, the system can include a bioreactor including the irradiation unit configured to irradiate contents of the bioreactor. The cell, decorated with a nanoparticle, can be used in a bioreactor to produce a chemical product when irradiated. Referring to FIG. 17, the system 10 can include a bioreactor 25 including an irradiation source 20. Bioreactor 25 can include a suspension 30 of the cell which is exposed to a precursor in the bioreactor. The precursor and/or the cell can be introduced into the bioreactor though inlet 40. Product can be removed through outlet 50.

The precursor can be a chemical species that is transformed by a biochemical reaction performed by the cell. The biochemical reaction performance can be enhanced by irradiation of the decorated cell. For example, carbon dioxide and glucose can be transformed into ethanol with a cadmium nanoparticle decorated yeast.

The most prominent biologically derived fuel around the world is ethanol. See, Short-Term Energy Outlook - U.S. Energy Information Administration (EIA), available at: https://www.eia.gov/outlooks/steo/ (accessed: 9th January 2020), which is incorporated by reference in its entirety. Currently, ethanol is mainly produced by fermentation of sugars from sugar cane or corn. See, Eagan, N. M., Kumbhalkar, M. D., Buchanan, J. S., Dumesic, J. A. & Huber, G. W. Chemistries and processes for the conversion of ethanol into middle-distillate fuels. Nat. Rev. Chem. 3, 223-249 (2019), which is incorporated by reference in its entirety. Enzymatic or thermocatalytic upgrading of synthetic gas has also resulted in ethanol production. See, Warner, E., Schwab, A. & Bacovsky, D. 2016 Survey of Non-Starch Alcohol and Renewable Hydrocarbon Biofuels Producers. (2015), which is incorporated by reference in its entirety. Ethanol currently used in the US is blended with gasoline levels of around 10% (compared to Brazil at 27%). See, Brazil: Biofuels Annual | USDA Foreign Agricultural Service, available at: https://www.fas.usda.gov/data/brazil-biofuels-annual-4 (accessed: 9th January 2020), which is incorporated by reference in its entirety. Adding ethanol to gasoline fuels has been shown to be beneficial for decreasing carbon monoxide and hydrocarbon emissions while increasing the octane number. See, Hsieh, W. D., Chen, R. H., Wu, T. L. & Lin, T. H. Engine performance and pollutant emission of an SI engine using ethanol-gasoline blended fuels. Atmos. Environ. 36, 403-410 (2002), and Agarwal, A. K. Biofuels (alcohols and biodiesel) applications as fuels for internal combustion engines. Progress in Energy and Combustion Science 33, 233-271 (2007), each of which is incorporated by reference in its entirety.

Artificially photosynthetic systems aim to chemically reduce carbon dioxide. See, Blankenship, R. E. et al. Comparing photosynthetic and photovoltaic efficiencies and recognizing the potential for improvement. Science 332, 805-9 (2011), which is incorporated by reference in its entirety. These processes can be imitated by hybrid inorganic-biological systems that have been developed to use light as a stimulus to drive product formation from carbon based molecules into liquid fuels. See, Guo, J. et al. Light-driven fine chemical production in yeast biohybrids. Science 362, 813-816 (2018), Sakimoto, K. K., Wong, A. B. & Yang, P. Self-photosensitization of nonphotosynthetic bacteria for solar-to-chemical production. Science 351, 74-7 (2016), Gust, D., Moore, T. A. & Moore, A. L. Solar Fuels via Artificial Photosynthesis. Acc. Chem. Res. 42, 1890-1898 (2009), Liu, C., Colón, B. C., Ziesack, M., Silver, P. A. & Nocera, D. G. Water splitting-biosynthetic system with CO₂ reduction efficiencies exceeding photosynthesis. Science 352, 1210-3 (2016), Liu, C. et al. Nanowire-Bacteria Hybrids for Unassisted Solar Carbon Dioxide Fixation to Value-Added Chemicals. Nano Lett. 15, 3634-3639 (2015), and Torella, J. P. et al. Efficient solar-to-fuels production from a hybrid microbial-water-splitting catalyst system. Proc. Natl. Acad. Sci. 112, 2337-2342 (2015), each of which is incorporated by reference in its entirety. US 8,227,237 describes engineered CO₂ fixing microorganisms.

Cadmium is a heavy metal with high toxicity even at very low exposure levels. Cadmium’s water solubility enables its circulation in the environment, mobility, and bioavailability. See, Nordic Council of Ministers Cadmium Review. (2003), which is incorporated by reference in its entirety. Cadmium can accumulate in the human body and cause kidney damage as well as lead to lung cancer and prostate cancer in high exposure settings. See, Fowler, B. A. Monitoring of human populations for early markers of cadmium toxicity: A review. Toxicol. Appl. Pharmacol. 238, 294-300 (2009), which is incorporated by reference in its entirety. Many techniques, such as chemical reduction, electrochemical treatment, ion exchange, precipitation, and absorption have been reported in an effort to clean up the cadmium waste. A biological system can be genetically engineered to uptake cadmium and remove the toxic metal from their environment. In certain embodiments, the biological system can include the yeast. The sequestered cadmium forms light-activatable nanoparticles that support biofuel synthesis. Yeast has been used as hyperaccumulators for heavy metals. Sun G. et al., Designing yeast as plant-like hyperaccumulators for heavy metals, Nature Communications (2019) 10:5080, which is incorporated by reference in its entirety. Yeast is also known to be a good model to study interactions with quantum dots. See, e.g., Pagano L. et al., In Vivo-In Vitro Comparative Toxicology of Cadmium Sulphide Quantum Dots in the Model Organism Saccharomyces cerevisiae (2019) Nanomaterials 9, 512 and Mei J. et al, The interactions between CdSe quantum dots and yeast Saccharomyces cerevisiae: Adhesion of quantum dots to the cell surface and the protection effect of ZnS shell, Chemosphere, October 2014, 112:92-99, each of which is incorporated by reference in its entirety. CN 101264 describes removing cadmium ion from waste water by waste beer yeast absorption, which is incorporated by reference in its entirety.

Microorganisms have been used for biomanufacturing due to their ability to produce higher value chemicals through growth in simple and inexpensive media. See, Sakimoto, K. K., Wong, A. B. & Yang, P. Self-photosensitization of nonphotosynthetic bacteria for solar-to-chemical production. Science 351, 74-7 (2016), and Mohd Azhar, S. H. et al. Yeasts in sustainable bioethanol production: A review. Biochem. Biophys. Reports 10, 52-61 (2017), each of which is incorporated by reference in its entirety. Certain microorganisms have been genetically engineered to convert renewable carbon sources into higher-value chemicals. Sakimoto, K. K., Wong, A. B. & Yang, P. Self-photosensitization of nonphotosynthetic bacteria for solar-to-chemical production. Science 351, 74-7 (2016), which is incorporated by reference in its entirety. Saccharomyces cerevisiae have been used in industrial settings due to the wide range of metabolites, biofuels, drug precursors, and flavors it can be engineered to produce. See, Jouhten, P. et al. Yeast metabolic chassis designs for diverse biotechnological products. Sci. Rep. 6, 29694 (2016), which is incorporated by reference in its entirety. US 8,465,954, US 9,752,164, and US 7,078,201 describe ethanol production by microorganisms, each of which is incorporated by reference in its entirety. Improved ethanol production has been observed in a certain mutant yeast. See, Hu, J. et al., Improved ethanol production in the presence of cadmium ions by a Saccharomyces cerevisiae transformed with a novel cadmium-resistance gene DvCRP1, Environmental Technology, 37:22, 2945-2952, which is incorporated by reference in its entirety. While the extensive genetic studies on this model organism have provided information to better understand and engineer yeast for product formation, the interplay between yeast physiology in an inorganic-biological hybrid remains poorly characterized. Additionally, the use of inorganic-biological hybrid systems can serve as a useful tool to toggle the metabolism of yeast in a rapid manner.

Light’s bioavailability, sustainability, and low cost render it a desirable stimulus in biological applications. Light has been used as an inducible and reversible stimulus to precisely garner biological responses. See, Zhao, E. M. et al. Optogenetic regulation of engineered cellular metabolism for microbial chemical production. Nature 555, 683-687 (2018), and Salinas, F., Rojas, V., Delgado, V., Agosin, E. & Larrondo, L. F. Optogenetic switches for light-controlled gene expression in yeast. Applied Microbiology and Biotechnology 101, 2629-2640 (2017), each of which is incorporated by reference in its entirety. Using a synthetic yeast system for light driven product formation can be an environmentally friendly, sustainable, and regenerable system. See Guo, J. et al. Light-driven fine chemical production in yeast biohybrids. Science 362, 813-816 (2018), which is incorporated by reference in its entirety. Chemically regulated systems have also been used to induce high levels of expression despite the limitations in causing undesired activation of physiological and signaling pathways and imprecise control over protein levels.

### Setup and Visual Characterization of the Hybrid System

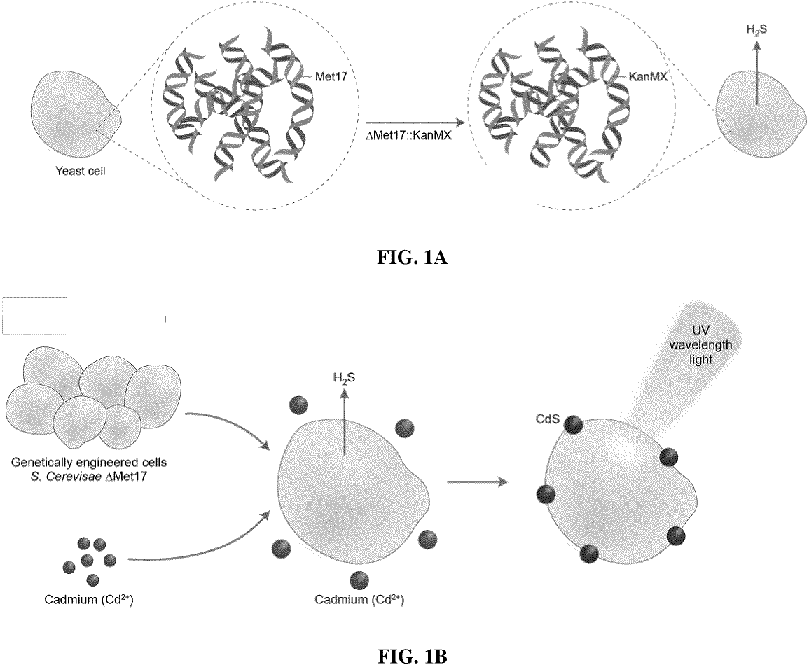

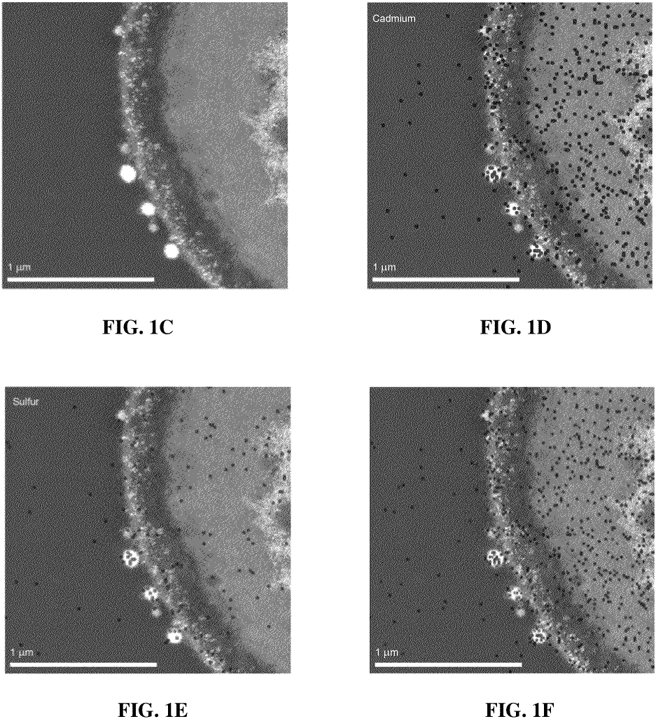

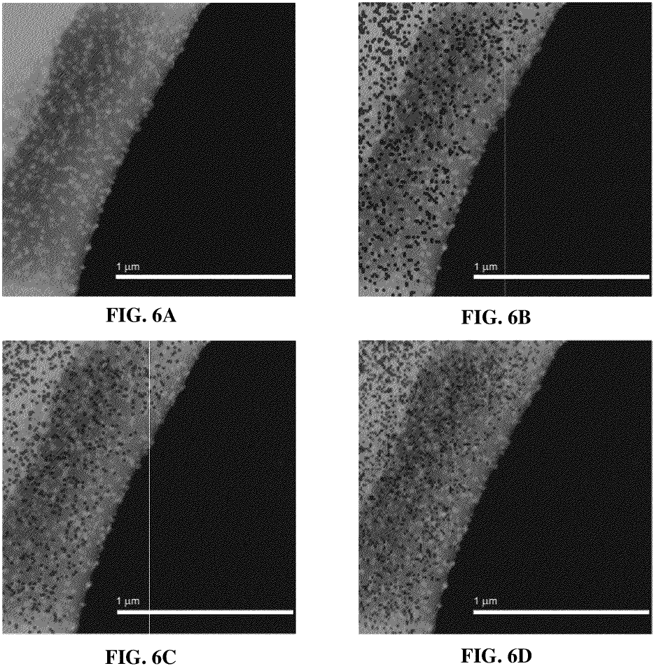

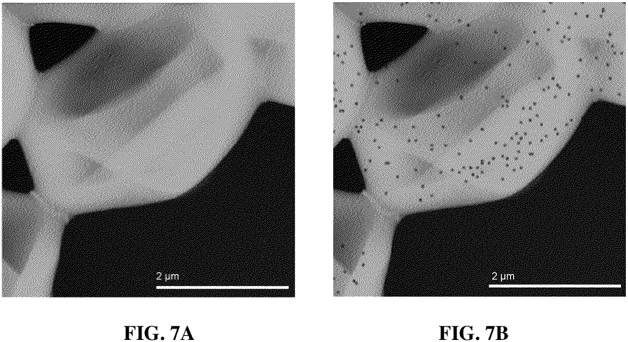

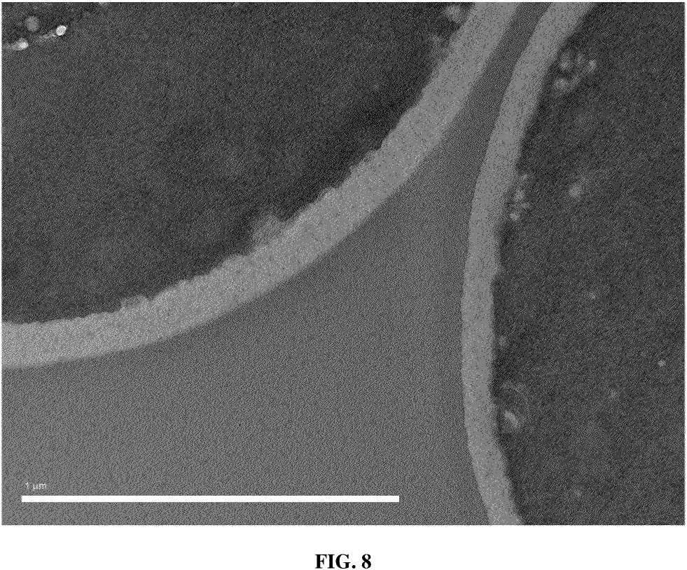

A yeast-nanoparticle hybrid system involving cadmium sulfide (CdS) nanoparticles was developed through genetic control for endogenous production of hydrogen sulfide (FIG. 1). Deletion of Met17 in Saccharomyces cerevisiae leads to an increase in hydrogen sulfide production, which is shuttled out of the cell. A pathway involved in thiol synthesis, Met17, was deleted from the Saccharomyces cerevisiae (S288C) strain, W303α, which led to an increase in hydrogen sulfide production (FIG. 1A). Treating this strain, W303α ΔMet17::KanMX (ΔMet17) with cadmium ions (Cd²⁺) results in CdS nanoparticle formation (FIG. 1B). Performing transmission electron microscopy (TEM) of the sample to physically characterize the system displayed that the exposure to Cd²⁺ causes the precipitation of CdS nanoparticles on the cell surface (FIG. 1C). Elemental mapping analysis measured the presence of cadmium (FIG. 1D) and sulfur (FIG. 1D) in the sample. Measuring both cadmium and sulfur simultaneously confirms the presence of CdS nanoparticles in element dense areas on the cell surface (FIG. 1F). Performing the same microscopy and analysis on an uncut ΔMet17 Cd²⁺ treated sample displays the same properties of cadmium and sulfur on the cell surface (FIG. 6). When W303α was treated with the same dose of Cd²⁺, no formation of nanoparticles was observed (FIG. 7, FIG. 8). CdS nanoparticles were extracted and isolated in order to characterize their excitation and emission properties. The nanoparticles have a maximum excitation at 350 nm with an emission of 415 nm.

### Transcriptomic Characterization of the Hybrid System

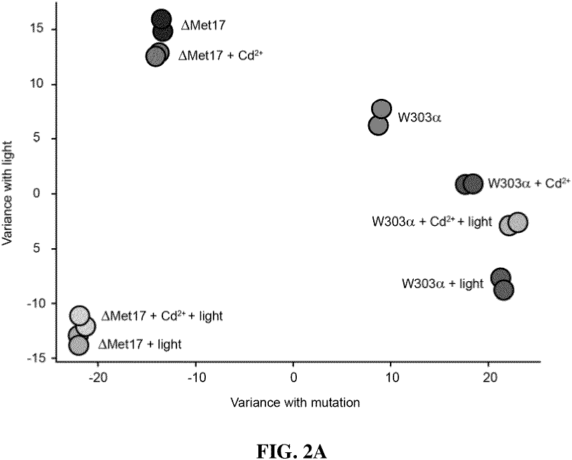

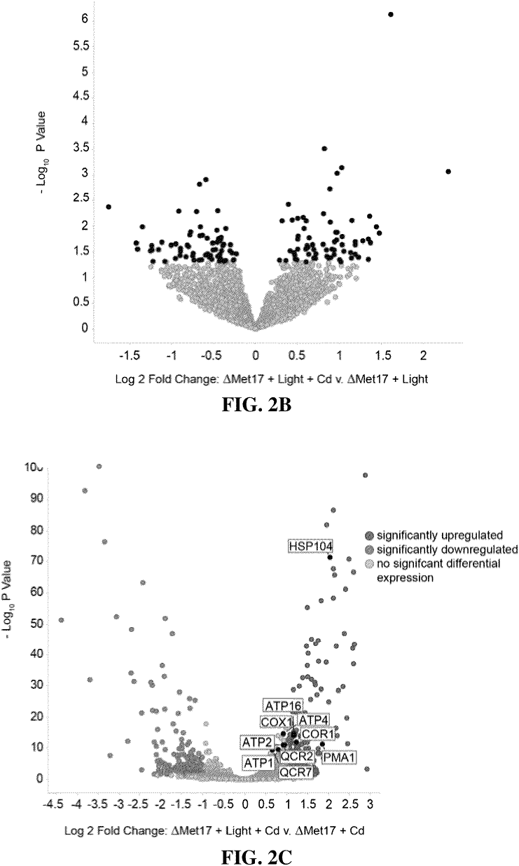

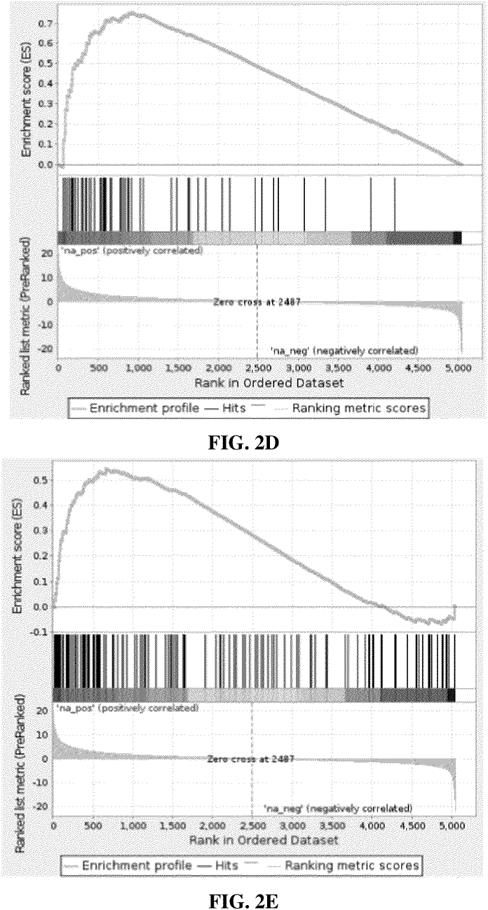

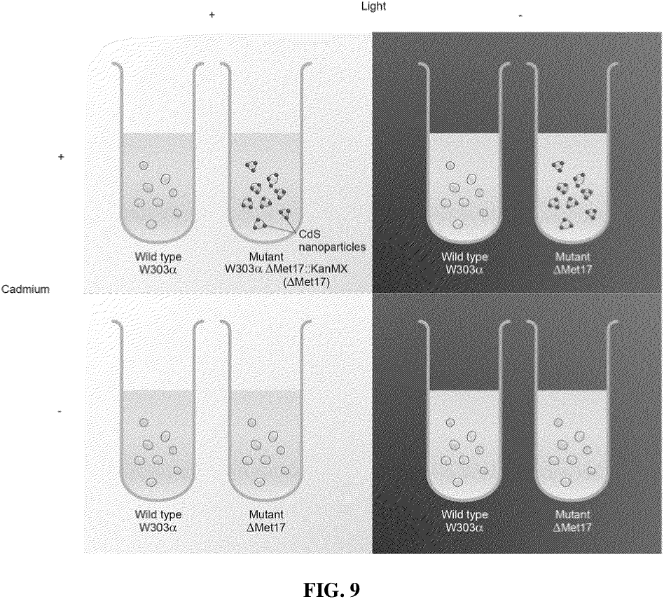

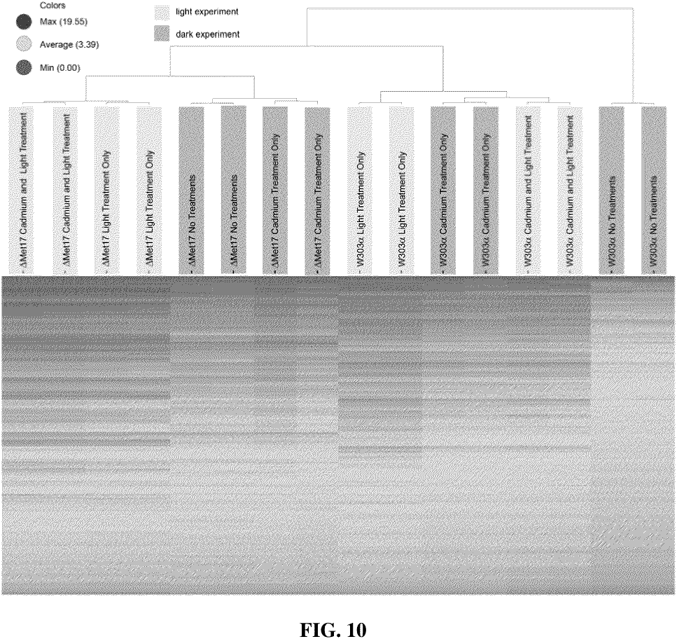

To elucidate the effects of the mutation, Cd²⁺ treatment, and ultraviolet light at 350 nm (light) treatment on gene expression, the transcriptome was characterized (FIG. 2). Both W303α and ΔMet17 were tested in the untreated, cadmium only, light only, and cadmium and light treatments (FIG. 9). Principal component analysis of the RNA sequencing data determined that the strongest variations in gene expression were caused by the implementation of the Met17 gene deletion and light treatment (FIG. 2A, FIG. 10). This analysis captured 48% of the variance due to the deletion and 15% of the variance due to light treatment. Further investigation was performed to reveal the effects due to both cadmium and light treatment through differential gene expression analysis. W303α samples were tested against each other, ΔMet17 samples were tested against each other, and ΔMet17 samples were tested against W303α. The analysis revealed an increase in gene transcripts with a nicotinamide adenine dinucleotide (NAD⁺) dependency due to the effects of CdS nanoparticles (FIG. 2B). Genes such as HST1, an NAD⁺ dependent gene, was found upregulated only in the ΔMet17 + Cd²⁺ + light treated sample when compared to the ΔMet17 + light sample (Table 1). Further investigation into the effects of light treatment revealed an increase in gene expression of transcripts involved in adenosine triphosphate (ATP) synthesis (ATP14, TIM11), as well as the electron transport chain, such as COX9 and QCR8 (FIG. 2C, Table 2). In order to delve deeper into the effects of light and cadmium treatment, gene set enrichment analysis (GSEA) was performed. Gene sets were chosen to include all genes involved in biofuel production, glycolysis, ATP production and regulation, fermentation, respiration, electron transport chain, and other metabolism related genes. Through GSEA, light treatment was shown to upregulate protein coding genes involved in proton pumping and mitochondrial electron transport chain. (FIG. 2D). An upregulation of genes involved in ATP synthesis and production were found. Genes involved in protein folding, cation transport, and hydrogen ion transmembrane transport were also found (Table 3). Upregulated genes involved in proton shuttling and proton transport suggest that electron transport and membrane potential were altered with a potential mechanism for shuttling electrons. Treatment with cadmium and precipitation of CdS nanoparticles was correlated with an upregulation of genes in involved in translation, DNA strand elongation, and proton transport (FIG. 2E). Genes involved in glycolysis were found to be upregulated. Certain genes, such as PMA1, COR1, and QCR3 were found to also be involved in glycolysis and production of metabolites used in respiration or fermentation (Table 4).

### Characterizing the Redox Properties of the Hybrid System

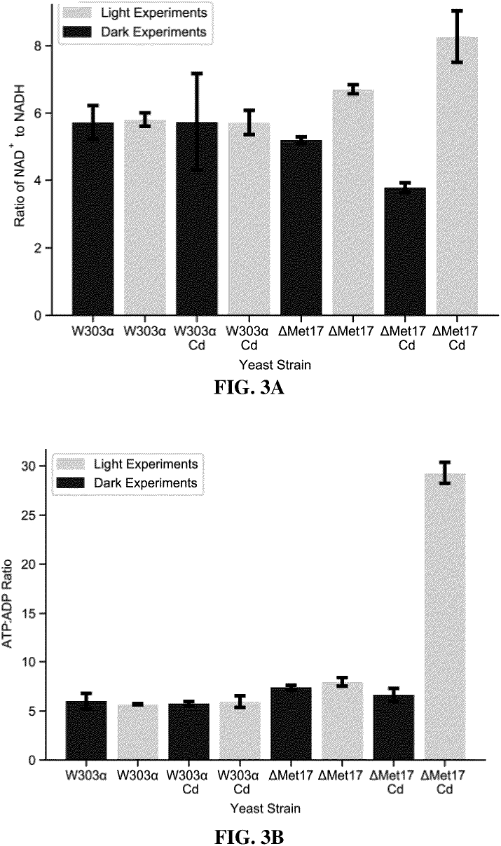

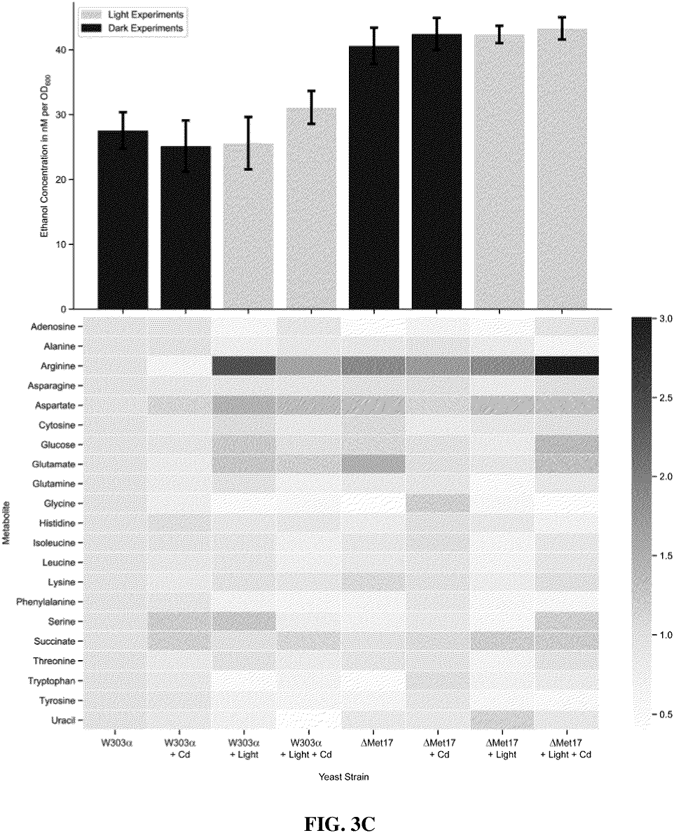

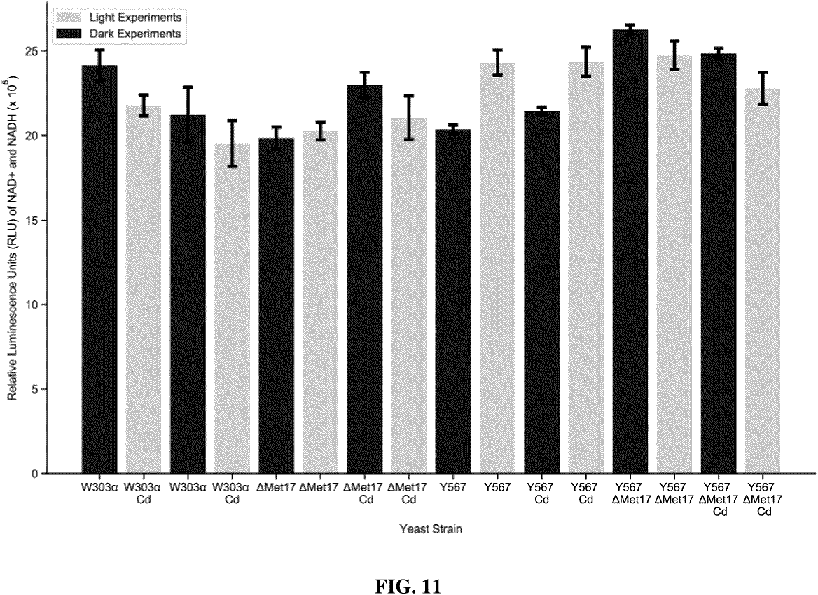

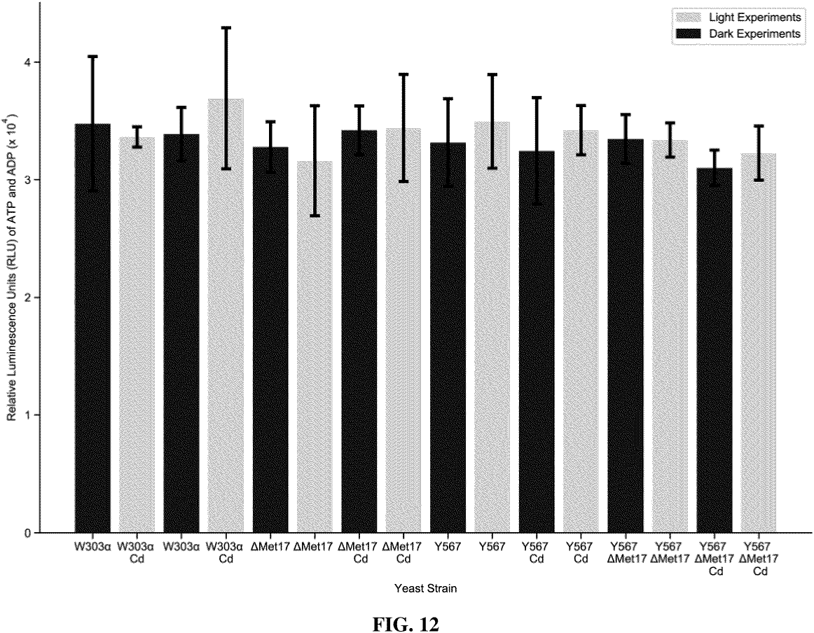

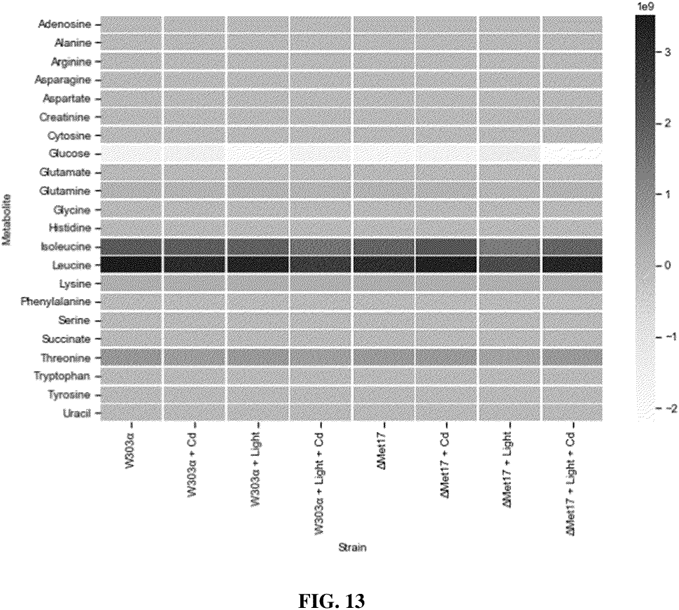

Further mechanistic investigation involved inquiry into metabolite concentrations in yeast strains. This was performed by characterizing the redox potential of cells through the NAD⁺:NADH ratio and the ATP:ADP ratio (FIG. 3). Intracellular NAD⁺ and NADH concentrations were measured and revealed an increase in the NAD⁺:NADH ratio in the ΔMet17 strain treated with Cd²⁺ and light (FIG. 3A). The total amount of NAD⁺ and NADH in each strain was similar (FIG. 11). As the effects of light displayed an increase in protein coding genes related to ATP production and regulation, intracellular ATP and ADP concentrations were measured. While the total amount of ATP and ADP was similar in each strain (FIG. 12), a marked difference in the ATP:ADP ratio was found in the ΔMet17 strain treated with Cd²⁺ and light (FIG. 8B). Liquid chromatography-mass spectrometry (LC-MS) was performed to identify substances within the media sample to determine the intake of nutrients by yeast strains (FIG. 8C). The relative rates of nutrient consumption remained similar across strains and treatment conditions comparted to the control. However, the nutrient consumption of ΔMet17 + Cd²⁺ + light was found to be lower than other treatments, particularly of arginine and glucose. Product formation through the change of redox potential in the cell was shown through measuring the concentration of ethanol, a biofuel of interest. An increase in the intracellular concentration of ethanol was found in the ΔMet17 strain treated with Cd²⁺ and light. An increase in ethanol concentration coupled with a decrease in glucose consumption could be indicative of increased efficiency in ethanol production when compared to wild-type. Intracellular metabolite concentrations were normalized to cell size, and flux analyses were normalized to growth rates.

### Implementing and Characterizing the Mutation in Another Yeast Strain

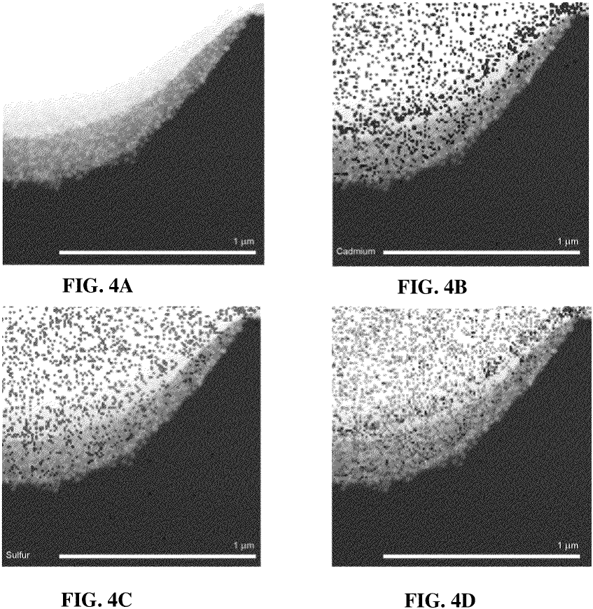

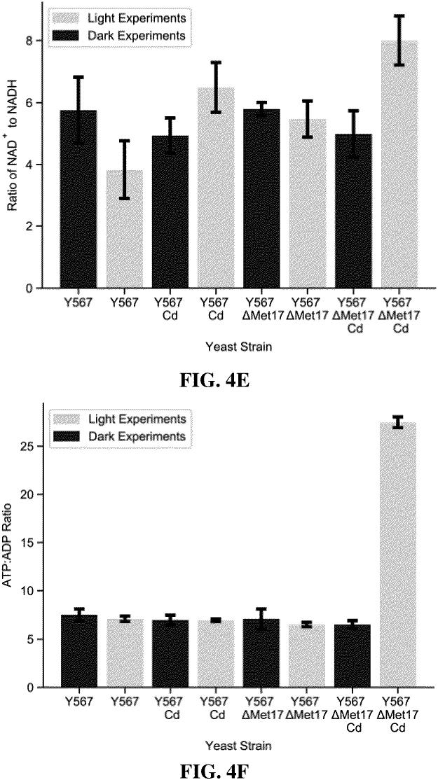

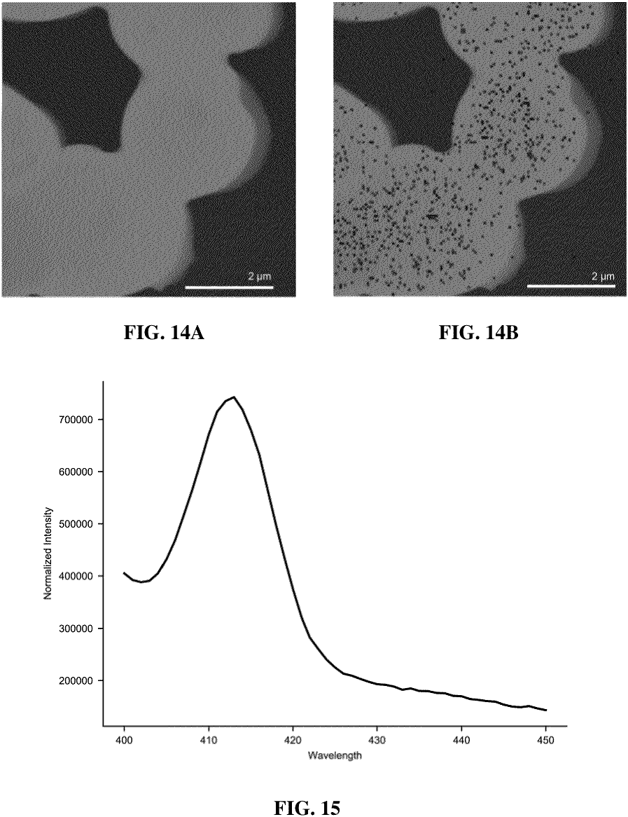

To verify the applicability of the Met17 deletion, and Cd²⁺ and light treatments, the mutation was implemented in another S. cerevisiae strain, Y567. Y567 was engineered to have an increased ethanol production capacity for the beer industry. After deletion, the behavior of the resultant strain, Y567 ΔMet17::KanMX (Y567 ΔMet17) was characterized (FIG. 4). When treated with the same dose of Cd²⁺ (10 µM) as W303α ΔMet17, Y567 ΔMet17 precipitates CdS nanoparticles on the cell surface (FIG. 4A). Elemental mapping analysis displays the presence of cadmium on the cell surface (FIG. 4B). Elemental mapping analysis measures the presence of sulfur on the cell surface (FIG. 4C). Mapping both sulfur and cadmium shows localization of the CdS nanoparticles (FIG. 4D). TEM and elemental analysis of the Y567 strain treated with the same dose of Cd²⁺ shows no precipitation of CdS nanoparticles, and is consistent with the behavior of W303α (FIG. 14). The CdS nanoparticles extracted from Y567 ΔMet17 had a maximum excitation at 350 nm with an emission at 415 nm (FIG. 15). Measurements of metabolite concentrations in Y567 ΔMet17 reveal an increased NAD⁺:NADH ratio (FIG. 4E) and an increase in ATP:ADP ratio (FIG. 9F). The total NAD⁺ and NADH levels as well as ATP and ADP levels remained similar in all strains independent of cadmium and light treatments (FIG. 11, FIG. 12).

### Increased Ethanol Production Through Hybrid System

In other biological-inorganic hybrid system, light harvesting semiconductor particles that were attached to the surface of the cell provided reducing agents to the metabolic processes. A similar mechanism of an excited electron from the CdS nanoparticle flowing to the metabolic processes in the yeast cell was hypothesized in this yeast-inorganic hybrid system. The transcriptomic upregulation in protein coding genes involved in the electron transport chain also supports this hypothesis (FIG. 3C).

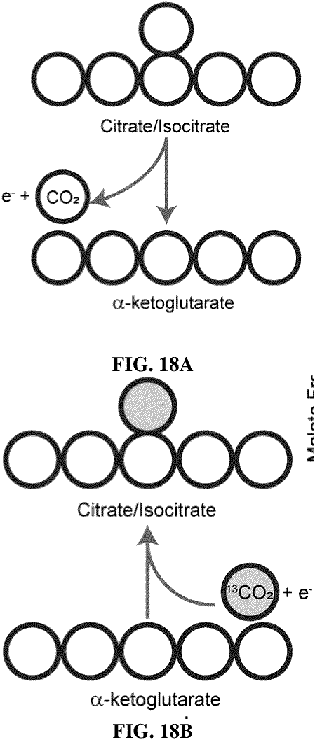

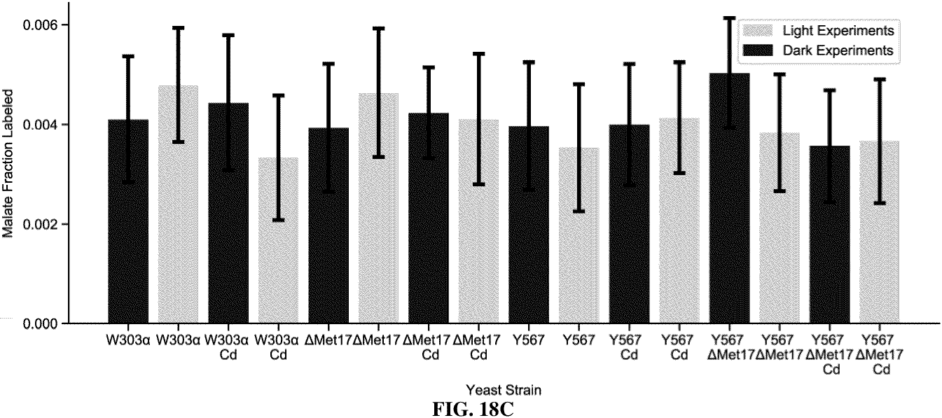

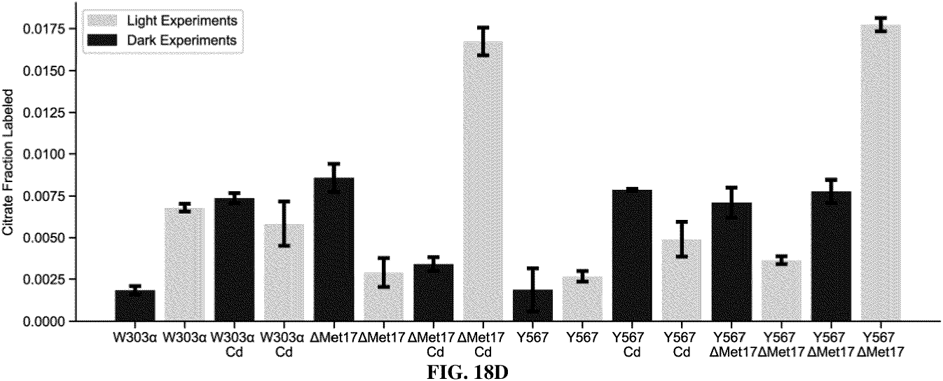

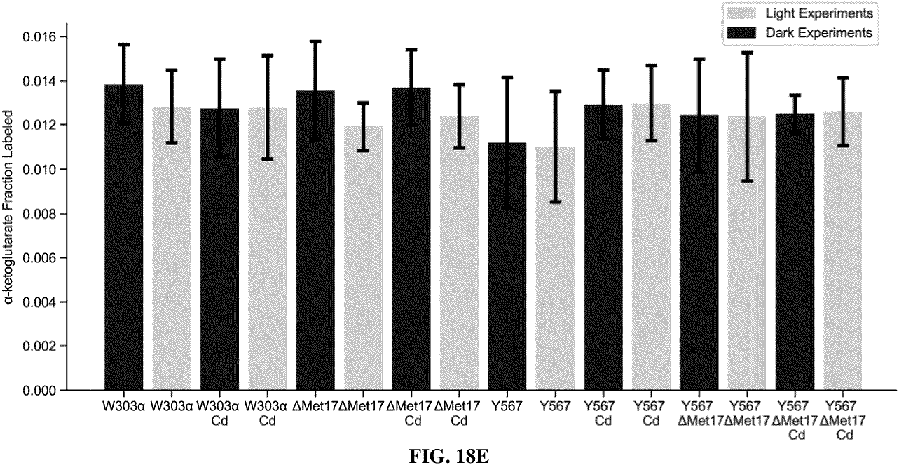

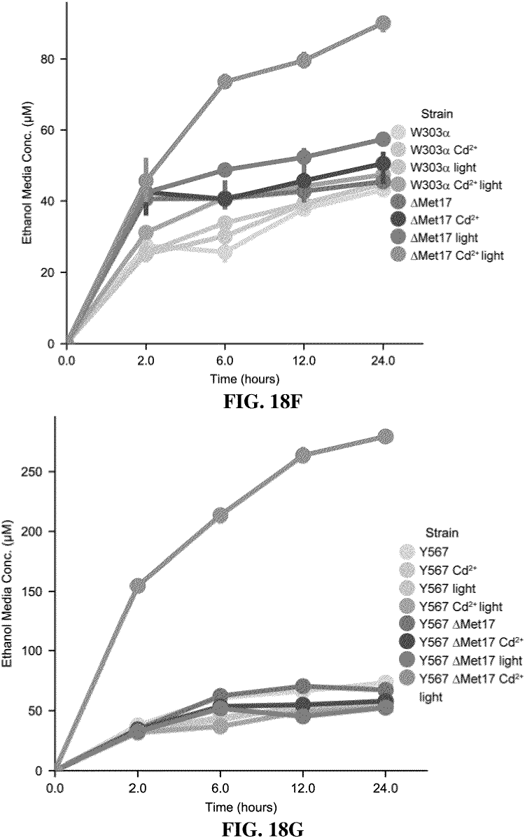

In other biological-inorganic hybrid system, light harvesting semiconductor particles that were attached to the surface of the cell provided reducing agents to the metabolic processes. A similar mechanism of an excited electron from the CdS nanoparticle flowing to the metabolic processes in the yeast cell was hypothesized in this yeast-inorganic hybrid system. The reductive carboxylation of the tricarboxylic acid (TCA) metabolite alpha-ketoglutarate into citrate has been reported as a redox responsive pathway that is engaged upon an increase in cellular electron donor availability in various biological systems. Thus, it was hypothesized that this pathway of CO₂ reduction can represent a potential light-stimulated reductive processes in the system (FIGS. 18A and 18B). To test this hypothesis, various strains of yeast were cultured with and without light in the presence of C¹³-labeled CO₂ and assessed label incorporation into citrate. Consistent with this hypothesis, it was found that the ΔMet17 W303α and ΔMet17 Y567 strain treated with Cd²⁺ and light had the greatest fraction of steady-state intracellular citrate labelled by CO₂ (FIG. 18D). Importantly, this increase in labelling was not seen in alpha-ketoglutarate, suggesting that the label on citrate is being incorporated through reductive carboxylation of alpha-ketoglutarate and not through other pathways of citrate synthesis (FIGS. 18C and 18E). The increase in M-5 citrate from is on the same order of magnitudes as previously seen reductive carboxylation pathways from alpha-ketoglutarate to citrate in mammalian cells.

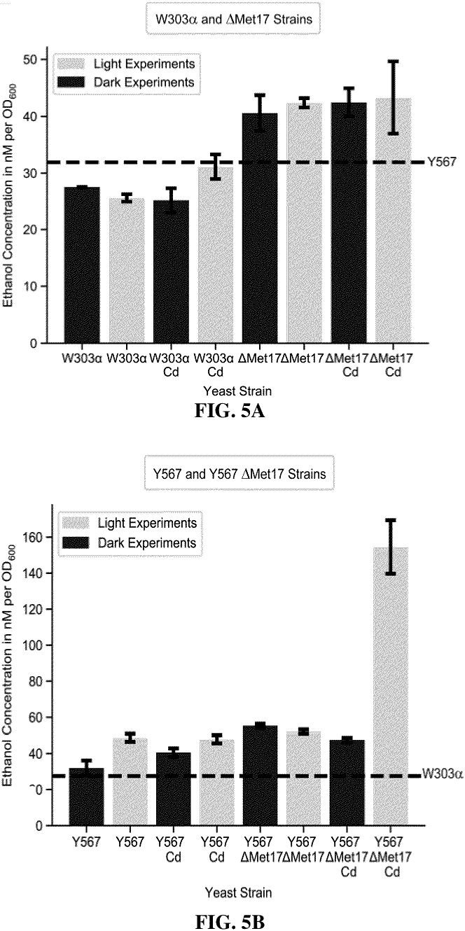

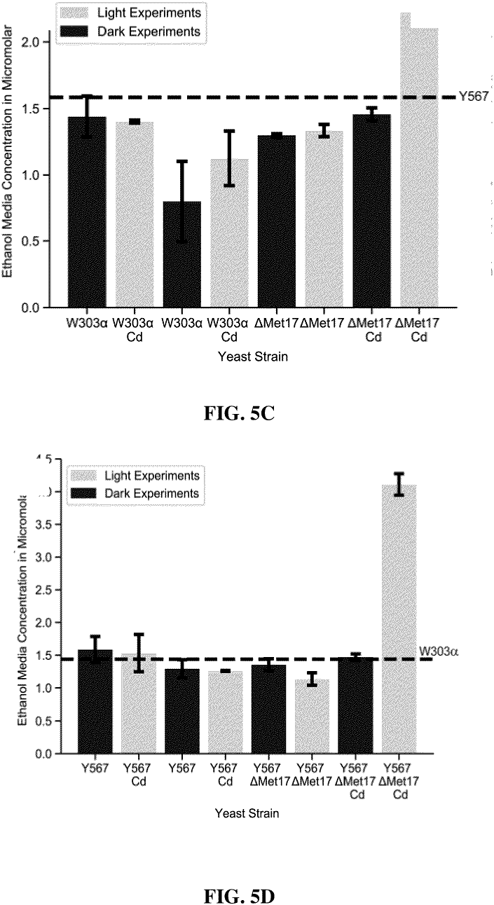

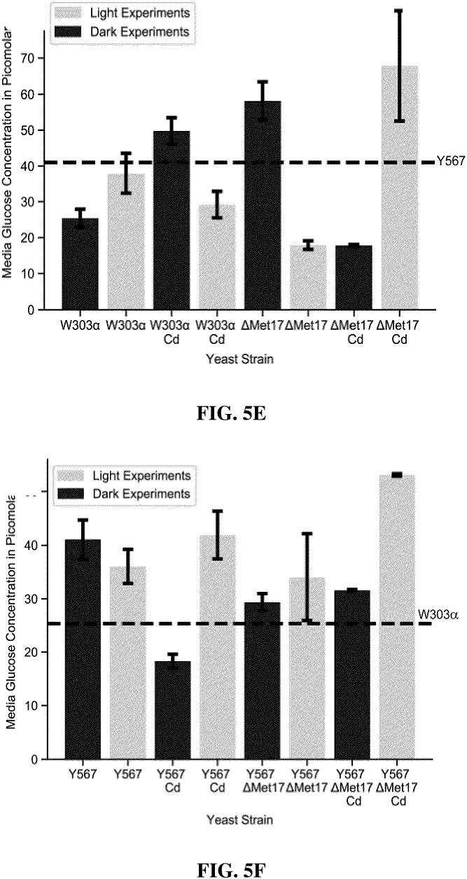

Within the yeast cell, yeast fermentation and metabolic processes can drive ethanol production, with a more reduced cell state favoring ethanol production as this pathway is driven by high NADH and allows electron disposal for NAD⁺ regeneration. An increase in intracellular ethanol concentration was found in the ΔMet17 strain treated with Cd²⁺ and light when compared with W303α. A 5-fold change in ethanol production was found in the Y567 ΔMet17 strain treated with Cd²⁺ and light when compared to Y567. The concentration of ethanol in the media was also measured to determine the change in ethanol secreted by the yeast strains over time. FIG. 18F shows concentration of ethanol in the supernatant media of W303α and ΔMet17 yeast strains after cadmium treatment and dark/light experiment. FIG. 18G shows concentration of ethanol in the supernatant media of Y567 and Y567 ΔMet17 yeast strains after cadmium treatment and dark/light experiment Ethanol concentration was higher in ΔMet17 treated with Cd²⁺ and light when compared to W303α (FIG. 18F). Similarly, the supernatant media of the Y567 ΔMet17 strain treated with Cd²⁺ and light was higher than the Y567 strain (FIG. 18G). The increase in ethanol in the mutant strains both inside the yeast cell and in its environment suggests a mechanism activated by CdS and light treatment that increases ethanol production. In order to test whether increased ethanol production was accompanied by increased glucose consumption, the glucose concentration of the media was examined. A lower glucose input was required by ΔMet17 (FIG. 18F) and Y567 ΔMet17 (FIG. 18G) strains for a higher yield of ethanol. The hybrid system’s increase in ethanol production and decrease in glucose consumption implies an offset cost in carbon balance that is supported by the CO₂ entry to the TCA cycle. This data, along with the observation that these strains are able to perform more reductive carboxylation in the presence of light, suggests this system can enable light-stimulated increased biofuel production from CO₂. Within the yeast cell, yeast fermentation and metabolic processes can drive ethanol production, with a potential increase due to the shuttling of electrons to regenerate NADH from NAD⁺. An increase in intracellular ethanol concentration was found in the ΔMet17 strain treated with Cd²⁺ and light when compared with W303α (FIG. 5A). This was exemplified with a 5-fold change in ethanol production in the Y567 ΔMet17 strain treated with Cd²⁺ and light when compared to Y567 (FIG. 5B). The concentration of ethanol in the media was also measured to determine the change in ethanol secreted by the yeast strains. Ethanol concentration was higher in ΔMet17 treated with Cd²⁺ and light when compared to W303α (FIG. 5C). Similarly, the supernatant media of the Y567 ΔMet17 strain treated with Cd²⁺ and light was 3-fold higher than the Y567 strain (FIG. 5D). The increase in the ethanol in the mutant strains both inside the yeast cell and in its environment suggests a mechanism caused by CdS and light treatment that increases ethanol production. In order to test whether or not certain strains took up more glucose, the glucose concentration of the media was tested. A lower glucose input was required by ΔMet17 (FIG. 5E, FIG. 3C) and Y567 ΔMet17 (FIG. 5F) for a higher yield of ethanol.

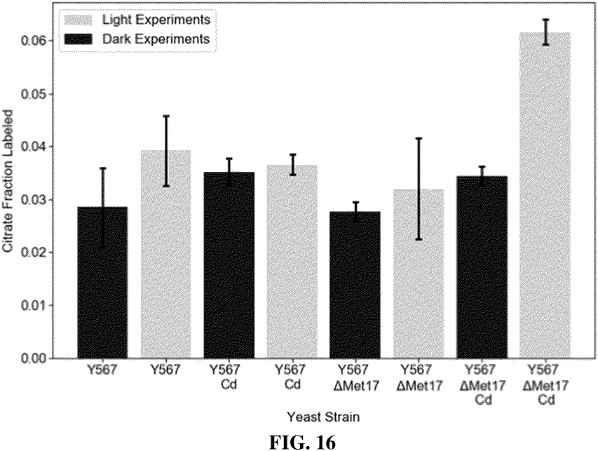

The change in redox potential, the need for a carbon source, and the decrease in glucose consumption lead us to hypothesize that the carbon source may be involved in the Calvin cycle. As part of photosynthesis, the Calvin cycle involves carbon dioxide fixation in the first stage. The second stage involves the donation of electrons from NADPH for the reduction of the carbon source. The net reaction of photosynthesis is photoactivation which releases electrons in the form of NADPH, which are then used to reduce carbohydrates. To test this hypothesis, radiolabeled carbon dioxide can be used. While only plants have rubisco, other organisms do have, carbon fixing enzymes, such as Isocitrate dehydrogenase. Alpha-ketoglutarate is converted to citrate with the input of carbon dioxide and consumes electrons in the form of NADPH. Citrate was observed as a proxy to see if the radiolabeled carbon dioxide is being fixed (FIG. 16). Radiolabeled carbon in citrate was increased in the mutant treated with light and cadmium, which implies that carbon dioxide fixation.

Development of an in-house inorganic-biological hybrid system has the potential to enable the production of higher value products. The production of propane-1,2-diol and propane-1,3-diol, that is already found in yeast, requires the reduction of NADH to NAD+. This work provides a platform to increase the production of fragrances, drug precursors, and other biofuels already produced by yeast. While a larger scale implementation will require the optimization of larger scale cultures and illumination sources, this hybrid-biological system can be tuned to fit various needs. The versatility of this system through the biological production of nanoparticles enables tuning of the yeast strain as well as the nanoparticle’s materials, size, and crystallinity. The intensity of ultraviolet light exposure via lamp at 3×10⁻⁶ W/m²/nm in a dark room is lower than atmospheric ultraviolet light levels at 10³ W/m²/nm. See, Climate Prediction Center -Stratosphere: UV Index: Nature of UV Radiation, available at: www.cpc.ncep.noaa.gov/products/stratosphere/uv_index/uv_nature.shtml (accessed: 9th January 2020), which is incorporated by reference in its entirety. The wavelength at which to excite the CdS nanoparticle can be tuned based on the size of the nanoparticle. The size of the nanoparticle can be controlled with the nutritional profile of the yeast through monitoring and control of hydrogen sulfide production. The genetic control of the biological production of nanoparticles can be implemented in various strains in addition to the two performed and discussed. A deeper understanding of the electron donation and transport mechanism can lead to further design improvements of the biological-hybrid system. This work provides a platform in which many tools can be tuned to enable efficient and economical production of valuable metabolites and products.

The development of an in vivo multicomponent hybrid system to modulate the redox properties of yeast cells with light and favor ethanol production illustrates how endogenous semiconductor CdS nanoparticle deposition can be used to alter the metabolic state of yeast for potential useful purposes. Here, it was demonstrate that the light induced yeast-CdS system can produce a 5.6x increase in ethanol production and a 9x increase in CO₂ incorporation. This system is adaptable to fit many applications, such as altering the nanoparticle’s material and/or optical properties, affecting CO₂ influx into yeast biomass, and the choice of yeast strain with or without engineered mutations can enable specific product formation.

The use of yeast in the manufacturing of high value pharmaceuticals, fragrances, and other renewable fuels should be amenable with this system, as many of these pathways are facilitated by a more oxidized NAD⁺/NADH ratio. The intensity of ultraviolet light exposure via lamp in the experiments at 3×10⁻⁶ W/m²/nm in a dark room is lower than atmospheric ultraviolet light levels at 10³ W/m²/nm (25), which implies that the light exposure needed to alter metabolic changes might be possible in the natural environment. While a larger scale implementation will require optimization of the culture size and illumination sources, the versatility of this system can be tuned to fit diverse needs. This hybrid system enables endogenous production of CdS nanoparticles, which, upon ultraviolet light treatment, changes the metabolic state of the yeast cell and drives product formation. The composite hybrid system minimizes the amount of handling necessary and integrates the tunability both from the semiconductor system and through the alteration of the metabolic state. This system provides a platform in which one can induce an organism to endogenously grow semiconductor material, collect light, alter redox properties of a living cell, and use the changes in redox potential to increase production of desired molecules, fix carbon dioxide, and reduce waste. This process can be tuned to enable efficient and economical production of other valuable metabolites and small molecule products.

The quantum yield of photosynthesis has been defined as the molar ratio between photons absorbed and oxygen released. Naturally and artificially photosynthetic systems have used the direct correlation between photon consumption and oxygen production as a measurement of efficiency. The hybrid system does not have such a direct correlation between photons absorbed and electrons accepted; however, differences in ethanol production via the hybrid system when compared with the wild-type yeast are seen. The system provides an increased production capacity and efficiency of ethanol.

### EXAMPLES

### Yeast Strain and Culture

Yeast strains W303α (S288C) and W303α ΔMet17 were available in the lab. Synthetically defined dropout media (SD) was made by dissolving 1.7 g/L yeast nitrogen base without amino acid and ammonium sulfate (YNB, Fischer), 5 g/L ammonium sulfate (Sigma), 0.6 g CSM-HIS-LEU-TRP-URA powder (MP Biologicals), 20 g/L glucose (Sigma), and 10 mL/L of 100X adenine hemisulfate stock (1 g/L, Sigma) in ddH₂O. 100X stocks of amino acids were created using the following: uracil (2 g/L, Sigma), histidine (5 g/L, Sigma), leucine (10 g/L, Sigma), and tryptophan (10 g/L, Sigma) were made in ddH₂O. They were subsequently filtered and sterilized prior to their use in supplementing cultures. Saccharomyces cerevisiae strain Y567 was acquired from ATCC, Strain: NRRL Y-567). Yeast strains were grown as previously described (19) and had a doubling time of ~140 minutes (Table 6).

Synthetically defined dropout medium was made by combining 1.7 g L⁻¹ yeast nitrogen base (YNB) without ammonium sulfate (Fischer) and amino acid amino acids. 5 g L⁻ ¹ ammonium sulfate (Sigma), 1.85 g 1⁻¹ dropout mix without cysteine and methionine (US Biological), 20 g L⁻¹ glucose (Sigma) and 10 ml L⁻¹ ×100 adenine hemisulfate stock (1 g 1⁻¹) (Sigma). CSM were combined by adding cysteine and methionine amino acids for a final concentration of 50 mg 1⁻¹ (Sigma). The dropout media and CSM (MP Biologicals) were adjusted to have a pH of 7.0 with addition of NaOH. Mixtures were stirred and filtered through a 0.22 µm filter top (EMD). YPD medium was made by combining 20 g L⁻¹ glucose (Sigma), 10 g L⁻¹ yeast extract, 20 g L⁻¹ peptone (Fisher) and were filter sterilized. Plates were made by adding 20 g L⁻¹ Bacto Agar (Fisher) and sterilization via autoclaving.

### Implementing ΔMet17 Mutation

The ΔMet17 mutation was implemented in both W303α and Y567. Met17 was knocked out inW303α and Y567 using the following primers for producing a deletion cassette KanMX:

| | |
|---|---|
| Name | Primer |
| del-Met-17-KanMX-fwd | TCAGATACATAGATACAATTCTATTACCCCCATCCATA CAGACATGGAGGCCCAGAATA (SEQ ID NO.: 1) |
| del-Met-17-KanMX-rev | AAGTAGGTTTATACATAATTTTACAACTCATTACGCAC ACCAGTATAGCGACCAGCATTC (SEQ ID NO.: 2) |
| seq-MET17-Kan-fwd | GGTTGGCAAATGACTAATTAAG (SEQ ID NO.: 3) |
| kanMX-rev | CAGTATAGCGACCAGCATTC (SEQ ID NO.: 4) |

Competent cells were created and the deletion cassette was transformed into yeast using a kit: Frozen EZ Yeast Transformation II (Zymo Research T2001).

Functionalizing CdS nanoparticles on the yeast cell surface and light experiments 20 mL of yeast culture was grown overnight in CSM media supplemented with all amino acids — leucine, tryptophan, uracil, and histidine. Cultures were grown at 30° C. shaking at 250 rpm. Overnight cultures were diluted down after fourteen hours of growth and resuspended in fresh CSM media supplemented with amino acids to an OD₆₀₀/mL of 0.2.

Cultures treated with cadmium ions (Cd²⁺, Sigma) were then treated with 10 uM cadmium for 4 hours, shaking at 250 rpm at 30° C. After cadmium ion treatment, cultures were subjected to UV wavelength light (380 nm, 3×10⁻⁶ W/m²/nm, 5.067 mW/cm²) for two hours. After treatment, cultures were spun down at 900xg for 4 minutes, the supernatant was removed, and immediately frozen using liquid nitrogen to preserve the native state.

Transmission Electron Microscopy (TEM) and elemental mapping analysis In all experiments, a non-expressing and non-treated wild-type control was used.

Sample slides of spheroplasted cells were prepared using from a MIT microscopy core. Samples were resuspended in 2 mL of fixative (3% glutaraldehyde, 0.1 M NaCacod pH 7.4, 5 mM CaCl₂, 5 mM MgCl₂, 2.5% sucrose) for 1 hour at 30° C. with gentle agitation (100 rpm). Cells were spun down at 900xg for 10 minutes.

For the osmium-thiocarbohydrazide-osmium staining: Cells were dispersed them embedded in a 2% ultra-low temperature agarose (made in ddH₂O). They were cooled and then cut into 1 mm³ cubes. Cubes were fixed in 1% OsO₄/ 1% potassium ferrocyanide in 0.1 M cacodylate/ 5 mM CaCl₂, pH 6.8 at room temperature for thirty minutes. Blocks were washed four times in ddH₂O for 1 minute each. Blocks were then transferred to 1% thiocarbohydrazide at room temperature for 5 minutes. Blocks were washed four times in ddH₂O for 15 minutes each.

Sample slides of non-spheroplasted cells were prepared in-house. Samples were spun down for 15 minutes at 900xg. The supernatant was removed and discarded. Samples were resuspended in 100 uL ddH₂O. 10 uL was suspended onto the center of the TEM copper grid. For the wash steps: 1 mL of ddH₂O was suspended on the hydrophobic side of parafilm.

Imaging was performed on a JEOL-2100 FEG microscope using the largest area size of the parallel illumination beam with a 100 micron condenser aperture. The microscope was operated at 200 kV with a magnification ranging from 2,000 to 600,000 for assessing the particle shape, particle size, and the atomic arrangement. The images were recorded via a Gatan 2kx2k UltraScan CCD camera. STEM imaging was performed via a high-angle annular dark field (HAADF) detector with a 0.5 nm probe size and 12 cm camera length in order to measure chemical information with energy dispersive X-ray spectroscopy (EDX). Elemental line scanning was performed using EDX via us of an 80 mm² X-Max detector (Oxford Instrument, UK).

### RNA Sequencing and Analysis

RNA extraction: Five OD₆₀₀ units of cells were collected. Cells were spun down and transferred to 2 mL screw-top Eppendorf tubes. The supernatant was removed then the cells were snap-frozen using liquid nitrogen. The cells were then resuspended in 400 uL TES buffer and 0.2 mL of 400 micron silica beads (OPS Diagnostics) were added. 400 uL of acid phenol (Life Technologies) was added and the samples were left to shake at 65 C for 45 minutes at 1100 rpm in a thermomixer (VWR). The samples were spun down at 14,000xg for 10 minutes. The supernatant was transferred (300 uL) was transferred to 1 mL of ice cold 100% ethanol and 40 uL of 3 M sodium acetate. The samples were mixed and incubated for sixteen hours overnight at 4 C. Pellets were aspirated and dried out in a hood then resuspended in 100 uL ddH₂O. They were resupsended on a shaker at 37 C for thirty minutes. A Qiagen RNeasy cleanup cut was used to clean up the sample (Qiagen 74106), with an additional step added to perform an on column DNase digestion (Qiagen 79254). Samples were then eluted with 50 uL of RNase free water. Samples were then transferred to the RNASequencing facility.

Samples were submitted to the BioMicro Center at MIT to be sequenced. All samples were extracted in biological duplicate, and technical triplicate. The entire experiment was done twice.

RNA sequencing data were aligned and summarized using STAR (version 2.5.3a), RSEM (version 1.3.0), SAMtools (version 1.3), and an ENSEMBL gene annotation of S. cerevisiae (3) was used. Differential gene expression analysis was performed with R (version 3.4.4), using DESeq (2_1.18.1). The resulting data were parsed then assembled with Tibco Spotfire Analayst (version 7.11.1). Gene sets for GSEA were procured from GO2MSIG database. All high quality GO annotations were used for Saccharomyces cerevisiae (S288c). Additional sets provided from the Amon Lab at MIT were also used. These sets are called “Gasch_ESR_Rep”, “Gasch_ESR_Ind”, and “TransposableElements”.

### Preparing Yeast Cell Lysate for Analysis

Yeast cells were thawed at room temperature and resuspended in 0.5 mg/mL 100T Zymolyase in 1 M Sorbital Citrate buffer at 1 mL per 10 OD₆₀₀. The resuspended culture was incubated at 30° C. for 1 hour. The resuspended culture was then spun down at 900xg for 15 minutes and the supernatant was removed and kept aside for further analysis. The spheroplasted pellet was resuspended in 3x the volume of the pellet in Yeast Lysis Buffer (Gold Bio, GB-178). The resuspended spheroplasted pellet was incubated on ice for 30 minutes. The lysed cells were centrifuged at 20,000xg for 30 minutes at 4° C. and the clear lysate was collected.

### Preparation of Media Supernatant for Analysis

After yeast cultures were grown, they were spun down at 900xg for 15 minutes. The supernatant was removed into new and separate tubes for metabolite analysis. The collected media supernatant was stored at -20° C.

### Cell Size Analysis

Cell size was measured using a coulter counter (Multisizer 3 Coulter Counter, Beckmann Coulter). Roughly 200,000 cells were analyzed for cell size in each condition after treatment for six hours - four hours of cadmium treatment and two hours of light/dark treatment.

### Measuring Intracellular NAD+/NADH Concentration

Intracellular NAD+ and NADH were measured using Promega’s NAD/NADH Glo Assay (Promega G9072) on the yeast lystates. This luminescent assay works by catalyzing reductase, in the presence of either the metabolite, to reduce a proluciferin reductase substrate to luciferin. The luciferin is proportional to the amount of NAD+ or NADH in the sample. This assay has a detection range of 10 nM to 400 nM.

### Measuring Intracellular ATP/ADP Concentration

ATP concentrated was measured using Promega’s CellTiter-Glo Luminescent Assay (Promega G7570) on the yeast lysates. The protocol was not altered. This luminescent assay uses beetle luciferin that is catalyzed to oxyluciferin by the presence of ATP. The tested sensitivity of this assay is between 10⁻²⁰ and 10⁻¹¹ moles of luciferase.

### Measuring Ethanol Concentration

Intracellular ethanol concentration was measured using Sigma’s Ethanol Assay Kit (Sigma MAK-076) kit on the yeast lysates. The ethanol concentration is determined by a coupled enzyme reaction, with a detection range of 10 uM to 10 nM per well.

### Liquid-chromatography Mass-spectrometry (LC/MS) Sample Preparation and Analysis

20 µL of yeast lysate was extracted with 180 µL of 80% methanol containing internal standards. The solution was vortexed for 30 seconds then spun down for ten minutes at 15,000 rpm at 4° C. Relative metabolites abundances were measured using a Dionex UltiMate 3000 ultra-high performance liquid chromatography system connected to a Q Exactive benchtop Orbitrap mass spectrometer equipped with an Ion Max source and a HESI II probe (Thermo Fisher Scientific). To quantify metabolite abundance from resulting, the chromatogram XCalibur QuanBroswer 2.2 (Thermo Fisher Scientific) was used in conjunction with the in-house retention time library of chemical standards.

### Measuring Extracellular Glucose Concentration

Extracellular glucose concentration was measured using Sigma’s High Sensitivity Glucose Assay Kit (Sigma MAK-181) on the yeast media supernatant. Glucose concentration is determined by a coupled enzyme assay resulting in a fluorometric readout (λₑₓ = 535 nm, λₑₘ = 587 nm) that is proportional to glucose concentration. The detection range of this assay is from 20-100 pmole/well.

### Measuring Labelled ¹³C-CO₂ incorporation into Intracellular Metabolites through GC-MS

After yeast growth through standard culture, light/dark experiments were performed in a sealed chamber. Within the chamber, ¹³C-CaCO₃ was reacted with HCl to produce ¹³CO₂. Localized atmospheric CO₂ was increased to 4%. The incorporation of the labeled CO₂ under different conditions was then tested via GC-MS.

500uL of yeast was pelleted and lysed in (4:3:8) methanol:0.88% KCl in water:dichloromethane. The samples were then spun at 15,000 g for 10 minutes, and the polar fraction was collected and dried down under nitrogen gas. Gas-chromatography coupled to mass spectrometry (GCMS) analysis was done as described previously (PMID: 24882210). Dried samples were derivatized with 20µL of methoxamine (MOX) reagent (ThermoFisher TS-45950) and 25µL of N-tert-butyldimethylsilyl-N-methyltrifluoroacetamide with 1% tert-butyldimethylchlorosilane (Sigma 375934). Following derivatization, samples were analyzed using a DB-35MS column ((30 m × 0.25 mm i.d. × 0.25 µm, Agilent J&W Scientific) in an Agilent 7890 gas chromatograph (GC) coupled to an Agilent 5975C mass spectrometer (MS). Data were analyzed and corrected for natural isotope abundance using in-house algorithms.

### Statistics

The experimental data are presented with the error bars representing standard deviation. All experiments were done in triplicate. All samples were blinded prior to experiments, resulting in all data being blinded prior to analysis. Statistics were performed using scipy and statsmodels. The chi-squared test and two-way ANOVA test were performed. P values are labelled as: ***P < 0.001, **P < 0.01, *P < 0.05.

TABLE 1

Log fold change gene expression pulling out the differential gene expression caused by the CdS nanoparticles. ΔMet17 + Cd2+ + light versus ΔMet17 + light were tested against each other. Gene names, log fold change values, and p-values are displayed

| Gene Name | Log Fold Change | Wald Test P Value |
|---|---|---|
| TAR1 | 0.316585499 | 2.93356E-52 |
| INO1 | 0.393514311 | 0.045219666 |
| BDH2 | -0.228409637 | 0.000199753 |
| PDC1 | 0.286817387 | 0.359416135 |
| ERG25 | 0.438602809 | 8.21841E-07 |
| ACS1 | 0.364412318 | 0.033803006 |
| IRA2 | -0.279149068 | 7.6757E-05 |
| STP3 | -0.257057051 | 0.096040881 |
| ADY2 | 0.467532171 | 5.96456E-08 |
| DRE2 | -0.352620156 | 3.66564E-25 |
| JLP1 | 0.581887841 | 0.024715494 |
| KGD2 | -0.304194935 | 0.012949936 |
| SSE2 | -0.266089633 | 5.90434E-20 |
| CWP1 | 0.89002328 | 3.12072E-63 |
| GAT2 | -0.451303045 | 1.71095E-35 |
| PIG2 | -0.297601864 | 1.30688E-16 |
| YEF3 | 0.571434423 | 2.22389E-10 |
| LGE1 | -0.439758406 | 0.006899376 |
| TSL1 | -0.378506512 | 0.000116195 |
| MET4 | -0.403292236 | 1.31778E-06 |
| KRE9 | -0.35297309 | 0.035825782 |
| RPS18A | 0.411722848 | 0.001381963 |
| RPL31A | 0.507425214 | 0.003605124 |
| RPL24A | 0.509307931 | 0.00090274 |
| HUA1 | -0.427963179 | 0.565425426 |
| RPS13 | 0.457667714 | 0.265225186 |
| LPD1 | -0.427009396 | 0.070030744 |
| SUR2 | -0.476726239 | 0.44404363 |
| YSY6 | 0.507622632 | 0.070289817 |
| RPL43B | 0.473806656 | 0.172731544 |
| RPN1 | -0.429161641 | 0.000484404 |
| DAL80 | -0.41560509 | 0.115844981 |
| GIS3 | -0.459162649 | 0.000120646 |
| ULS1 | -0.373723337 | 3.73215E-06 |
| ARO10 | -0.799158441 | 0.194060763 |
| PET20 | -0.593334678 | 0.017316265 |
| KNS1 | -0.409477856 | 0.008209141 |
| HAA1 | -0.392004727 | 0.003248049 |
| MEP2 | -0.670443118 | 0.844611412 |
| RPS9B | 0.63993678 | 0.047258617 |
| SFT2 | -0.416606195 | 0.000775643 |
| UME6 | -0.576219559 | 0.000167648 |
| MRPS5 | 0.628380397 | 0.000357977 |
| MIX14 | 0.614096785 | 9.93519E-06 |
| MPM1 | -0.513160267 | 0.035375452 |
| RPL12B | 0.608854514 | 0.002168024 |
| VPS27 | -0.457363253 | 0.249517763 |
| TOS8 | -0.35778191 | 0.000340163 |
| PUF3 | -0.453301077 | 0.589481418 |
| MEP1 | -0.703155825 | 0.689958106 |
| REX2 | 0.505098981 | 1.85478E-05 |
| SPT4 | 0.60626304 | 0.610491472 |
| TDH2 | 0.976168323 | 0.420807637 |
| RPL14A | 0.695087541 | 0.084209561 |
| SDH3 | -0.663704792 | 0.013326403 |
| RPL41B | 1.611282088 | 0.234679145 |
| YIP3 | -0.898061543 | 0.723182711 |
| ENO2 | 0.823894442 | 0.001508733 |
| RPL16B | 0.6005835 | 0.138652032 |
| SDP1 | -0.534996369 | 5.65913E-06 |
| PTC3 | -0.553134711 | 0.190948207 |
| CDS1 | 0.876359493 | 0.902116534 |
| CAF120 | -0.63159292 | 0.160486145 |
| UGA4 | -0.50883639 | 0.430344122 |
| EXO1 | 0.813890546 | 0.001181687 |
| MSB4 | 0.496464116 | 0.019087769 |
| NET1 | -0.600092224 | 0.34735793 |
| SRL3 | 0.72143486 | 0.79650161 |
| TOK1 | -0.521132378 | 0.183862911 |
| NHP10 | -0.572704465 | 0.657074626 |
| RCR2 | -0.660593021 | 0.001034293 |
| RSM23 | 0.717105604 | 0.299376275 |
| ACE2 | 0.894511096 | 0.063939462 |
| NOP1 | 1.056500513 | 0.294113039 |
| TIF1 | -0.902324128 | 0.000515909 |
| NMA111 | -0.732449122 | 0.011827918 |
| ERD2 | 0.810005821 | 0.919542897 |
| SSM4 | -0.916692357 | 0.055119613 |
| RPB7 | 0.683677225 | 0.707194342 |
| GUS1 | 1.032081398 | 0.011917773 |
| FAU1 | 0.69625037 | 0.52104234 |
| MSS2 | -0.895652038 | 0.019147313 |
| ADD37 | 0.849603986 | 0.001184318 |
| TCB2 | 0.635582755 | 0.796693929 |
| CTR9 | -0.74423745 | 0.848467387 |
| TSR4 | -0.674718951 | 0.501240136 |
| KRR1 | 0.95817913 | 0.401397986 |
| RPL33A | 0.917343725 | 0.083293426 |
| RPS22A | 0.976411204 | 0.904347866 |
| MNT4 | 0.8540384 | 0.568657495 |
| RPF2 | -0.832836868 | 0.010504341 |
| ATP23 | -0.955623416 | 0.826679846 |
| ASTI | -1.08528461 | 0.255710519 |
| DMA1 | -0.816767181 | 0.220221411 |
| AAH1 | 1.146406504 | 0.383880968 |
| OST5 | 0.968456005 | 0.33164699 |
| IMP4 | ⁻0.783855887 | 0.014103337 |
| HEL2 | -0.64503454 | 0.049191843 |
| WSS1 | 0.941029355 | 0.975781693 |
| GLY1 | -1.349517541 | 0.145553002 |
| GEP3 | 0.944575092 | 0.756124404 |
| SWD1 | 0.941870129 | 0.300345848 |
| SKI2 | -0.751460348 | 0.377579773 |
| MPS3 | -1.008106684 | 0.448936774 |
| PKH3 | -0.773382811 | 0.288121693 |
| RIM2 | 1.037990725 | 0.320220036 |
| ILS1 | -0.952154763 | 0.308315846 |
| RBL2 | 1.006317226 | 0.039032417 |
| CAF130 | -0.897127098 | 0.340951539 |
| RPL22B | 1.037657946 | 0.242623893 |
| UTP10 | 1.153242764 | 0.476579373 |
| ARP7 | 1.136648022 | 0.088920876 |
| ERG26 | 1.190852176 | 0.779702859 |
| MRH4 | 1.279302519 | 0.454755115 |
| DDI2 | 2.300995954 | 8.86046E-06 |
| NRM1 | 1.477760327 | 0.390205386 |
| AIM4 | 1.442695221 | 0.882248011 |
| MRE11 | 1.023299034 | 0.060650778 |
| DAK2 | -1.406147373 | 0.110060258 |
| SYM1 | 1.118469015 | 0.588871853 |
| DCN1 | 1.234900734 | 0.194496375 |
| GAS3 | 1.19533281 | 0.788984572 |
| SUA5 | 1.191721783 | 0.592949197 |
| HEM4 | -1.241552917 | 0.115136363 |
| MDM1 | 0.900628497 | 0.703963487 |
| FIRI | -1.257333078 | 0.311128612 |
| OSW1 | 1.361725474 | 0.000390617 |
| PEA2 | 1.344622629 | 0.597453941 |
| BUD8 | -1.423420634 | 0.584075378 |
| PHO5 | -1.156783335 | 0.094547619 |
| AVT2 | 1.230982833 | 0.345595078 |
| ATP10 | -1.2246438 | 0.584090756 |
| RSA4 | 1.346038096 | 0.841032706 |
| RP17B | 1.370131812 | 0.60531762 |
| RAD53 | -1.751017412 | 0.681744345 |

TABLE 2

Log fold change gene expression pulling out the differential gene expression caused by the UV light treatment. ΔMet17 + Cd²⁺ + light versus ΔMet17 + Cd²⁺ were tested against each other. Gene names, log fold change values, and p-values are displayed

| Gene Name | Log Fold Change | Wald Test Value | Adjusted P Value (Benjamini-Hochberg) |
|---|---|---|---|
| HXT6 | 1.956360444 | 1.13587E-85 | 1.20288E-82 |
| HSP26 | 1.507861553 | 1.72662E-43 | 3.42842E-41 |
| HSP30 | 1.2171177 | 5.28104E-25 | 4.86315E-23 |
| SPI1 | 1.383509704 | 1.57394E-35 | 2.38114E-33 |
| HSP82 | 2.401990489 | 1.28456E-64 | 5.83009E-62 |
| FIT2 | 1.164172255 | 1.68603E-31 | 1.91304E-29 |
| HSC82 | 1.823976227 | 6.448E-61 | 2.56066E-58 |
| BTN2 | 2.484071381 | 1.62463E-74 | 1.14699E-71 |
| TDH3 | 1.296717654 | 1.12007E-32 | 1.39548E-30 |
| HSP104 | 2.036397981 | 4.21151E-75 | F 3.345E-72 |
| HAP4 | 1.20550844 | 2.94919E-24 | 2.43366E-22 |
| STI1 | 2.119467349 | 1.14353E-61 | 4.84397E-59 |
| GCN4 | 1.225903712 | 1.03528E-24 | 9.26503E-23 |
| HSP42 | 1.491985569 | 1.94095E-39 | 3.33319E-37 |
| TEF2 | 1.475221009 | 7.64279E-46 | 1.67456E-43 |
| NCE102 | 1.486833182 | 1.00959E-58 | 3.77348E-56 |
| AI1 | 1.622861063 | 1.51389E-21 | 1.0931E-19 |
| PIC2 | 1.209031549 | 1.18393E-24 | 1.04482E-22 |
| HSP78 | 1.594465432 | 3.6756E-48 | 9.3419E-46 |
| PYC1 | 1.004724491 | 6.12588E-15 | 2.9266E-13 |
| SSA4 | 1.942580683 | 1.20608E-40 | 2.25395E-38 |
| ZRT1 | 2.111514054 | 2.11975E-90 | 2.69378E-87 |
| SIS1 | 2.114090321 | 2.50332E-71 | 1.59061E-68 |
| SSC1 | 1.308340892 | 4.77304E-22 | 3.56799E-20 |
| HSP10 | 1.093157248 | 7.45922E-14 | 3.18093E-12 |
| STF2 | 1.136755594 | 2.42114E-24 | 2.05119E-22 |
| HRK1 | 1.38349281 | 1.34636E-21 | 9.83305E-20 |
| CWP2 | 1.41755554 | 1.37083E-18 | 8.88804E-17 |
| STP4 | 2.874222253 | 9.0678E-102 | 1.92056E-98 |
| BDF2 | 1.497100852 | 7.81854E-36 | 1.24198E-33 |
| HTA1 | 1.342417439 | 2.35495E-27 | 2.33802E-25 |
| FES1 | 2.373704712 | 3.19922E-50 | 9.23993E-48 |
| SSE1 | 2.146770206 | 3.20839E-69 | 1.69884E-66 |
| IRA2 | 1.671009588 | 6.26667E-34 | 8.47586E-32 |
| YHB1 | 1.217842998 | 6.05111E-12 | 2.12424E-10 |
| CDC48 | 1.112884625 | 1.88546E-20 | 1.31651E-18 |
| DRE2 | 1.104358024 | 1.26593E-14 | 5.8288E-13 |
| HSP60 | 1.688338712 | 1.22068E-46 | 2.87267E-44 |
| ENO1 | 1.170534894 | 7.78197E-17 | 4.33742E-15 |
| MDJ1 | 1.443445729 | 5.19665E-24 | 4.23327E-22 |
| BAT2 | 1.056547681 | 1.04485E-14 | 4.91777E-13 |
| SSA1 | 1.76556869 | 6.32321E-41 | 1.2175E-38 |
| COX1 | 1.174773426 | 1.84439E-16 | 9.93157E-15 |
| APJ1 | 1.681666448 | 4.01894E-33 | 5.32008E-31 |
| GAP1 | 1.588740009 | 5.55607E-35 | 8.21006E-33 |
| TDH1 | 1.299616565 | 5.36105E-24 | 4.31191E-22 |
| MPC2 | 1.821922021 | 9.48664E-32 | 1.11626E-29 |
| CPR6 | 1.752872727 | 9.59266E-48 | 2.3443E-45 |
| DSE4 | 1.319798553 | 1.67172E-16 | 9.0787E-15 |
| RGI1 | 1.189270919 | 1.44934E-16 | 7.93887E-15 |
| MBF1 | 1.034957585 | 3.40785E-11 | 1.08811E-09 |
| SSE2 | 1.128539079 | 7.27249E-16 | 3.72657E-14 |
| PIN3 | 1.068582487 | 1.13751E-12 | 4.32798E-11 |
| CYT1 | 1.076396952 | 7.25928E-17 | 4.0819E-15 |
| AI2 | 2.344313048 | 1.30318E-32 | 1.59239E-30 |
| ATP16 | 1.169486335 | 3.26552E-17 | 1.88628E-15 |
| GLK1 | 1.119295036 | 2.55255E-11 | 8.27496E-10 |
| HXT7 | 1.726284159 | 6.01589E-30 | 6.47881E-28 |
| PDA1 | 1.167046412 | 7.345E-18 | 4.40284E-16 |
| AATI | 2.594379326 | 3.83466E-70 | 2.21504E-67 |
| LYS14 | 1.20878494 | 1.56441E-17 | 9.28998E-16 |
| ISF1 | 1.05641019 | 1.41961E-15 | 7.10252E-14 |
| GLN1 | 1.241916599 | 4.48058E-13 | 1.7683E-11 |
| EIS1 | 1.184453062 | 4.42306E-11 | 1.40521E-09 |
| PRC1 | 1.22656479 | 2.53359E-18 | 1.5939E-16 |
| TMA19 | 1.007216747 | 6.14189E-11 | 1.90369E-09 |
| TRR1 | 1.188474772 | 5.86958E-14 | 2.5371E-12 |
| COR1 | 1.232027998 | 2.65555E-14 | 1.19669E-12 |
| NDI1 | 1.338976739 | 1.2603E-13 | 5.20988E-12 |
| SGT2 | 1.576691742 | 2.1956E-28 | 2.28702E-26 |
| UGA1 | 1.050061414 | 3.21521E-11 | 1.03179E-09 |
| LAT1 | 1.353517596 | 1.85588E-20 | 1.31025E-18 |
| HXK1 | 2.181407991 | 7.97949E-46 | 1.69006E-43 |
| PCL5 | 1.009358852 | 5.91082E-13 | 2.30413E-11 |
| CTR1 | 2.002267708 | 1.6279E-27 | 1.64185E-25 |
| LST8 | 1.251491901 | 5.10932E-12 | 1.83416E-10 |
| GUT2 | 1.158053523 | 4.87298E-09 | 1.15966E-07 |
| OYE2 | 1.556792777 | 2.08748E-15 | 1.0203E-13 |
| lXR1 | 1.339268285 | 1.99771E-16 | 1.06668E-14 |
| UBC4 | 1.202049736 | 5.73344E-10 | 1.56353E-08 |
| YDJ1 | 1.795124378 | 6.39849E-22 | 4.72744E-20 |
| BAP3 | 1.235836361 | 1.46427E-09 | 3.7516E-08 |
| UTH1 | 1.52628255 | 3.78861E-12 | 1.3835E-10 |
| ALT1 | 2.614372661 | 2.01532E-46 | 4.57334E-44 |
| SCW4 | 2.232934737 | 2.91814E-31 | 3.25296E-29 |
| YAK1 | 1.097010779 | 3.95609E-10 | 1.13742E-08 |
| COM2 | 1.575920311 | 3.49822E-18 | 2.15803E-16 |
| THI4 | 1.361045341 | 1.35588E-08 | 2.99141E-07 |
| A14 | 1.305419711 | 2.54952E-11 | 8.27496E-10 |
| VHR1 | 1.079155961 | 6.17695E-10 | 1.66306E-08 |
| DAL80 | 1.410660943 | 4.97601E-14 | 2.16559E-12 |
| GIS3 | 1.274387956 | 3.18996E-11 | 1.02888E-09 |
| ACA1 | 2.600064495 | 5.24747E-40 | 9.5264E-38 |
| RTK1 | 1.235778303 | 5.44983E-12 | 1.93454E-10 |
| SNQ2 | 1.134039254 | 6.26568E-09 | 1.46368E-07 |
| REE1 | 2.573960349 | 4.03823E-45 | 8.27707E-43 |
| CAB2 | 1.41238652 | 3.34243E-17 | 1.91331E-15 |
| PMA1 | 1.858406007 | 1.2627E-13 | 5.20988E-12 |
| MEP2 | 2.17630025 | 5.75199E-19 | 3.76785E-17 |
| MTL1 | 1.122794847 | 1.24145E-07 | 2.35468E-06 |
| LEU2 | 1.271763909 | 8.44812E-13 | 3.25329E-11 |
| RPL35B | 1.195619715 | 0.003882638 | 0.023273853 |
| YAP1801 | 1.150034571 | 3.84444E-09 | 9.32349E-08 |
| PSA1 | 1.030985195 | 1.90609E-06 | 2.90439E-05 |
| ARB1 | 1.247153863 | 5.57226E-11 | 1.74414E-09 |
| ASN1 | 1.794286386 | 2.97236E-18 | 1.8516IE-16 |
| TP03 | 1.076871609 | 1.40377E-06 | 2.22988E-05 |
| AI5_BETA | 1.175796615 | 2.5909E-07 | 4.78563E-06 |
| RTS3 | 1.316017431 | 4.00067E-10 | 1.13992E-08 |
| FMS1 | 1.111117259 | 8.941E-08 | 1.74803E-06 |
| COQ9 | 1.076995916 | 2.32442E-09 | 5.88422E-08 |
| PDB1 | 1.232154613 | 1.07959E-06 | 1.78639E-05 |
| PST2 | 1.00680877 | 1.76335E-08 | 3.78524E-07 |
| HOM2 | 1.345379852 | 6.0056E-07 | 1.04834E-05 |
| ARG4 | 1.535205092 | 2.66015E-09 | 6.62846E-08 |
| ERO1 | 2.201900403 | 2.06005E-16 | 1.0908E-14 |
| EMI2 | 1.616886642 | 1.67517E-17 | 9.85558E-16 |
| CTH1 | 1.664419817 | 7.80902E-16 | 3.96948E-14 |
| EAF7 | 1.107144149 | 6.91746E-07 | 1.19439E-05 |
| VHS1 | 1.456331198 | 4.43101E-12 | 1.60884E-10 |
| OLA1 | 1.353739166 | 2.12293E-13 | 8.59177E-12 |
| TSA2 | 1.600127684 | 6.91418E-16 | 3.57177E-14 |
| MSN4 | 1.196137854 | 6.60251E-06 | 8.92604E-05 |
| MCM1 | 1.305831308 | 2.51186E-08 | 5.34951E-07 |
| ZPR1 | 1.875999842 | 6.03195E-09 | 1.41428E-07 |
| UTR1 | 1.066006468 | 2.35746E-09 | 5.94417E-08 |
| YRO2 | 2.436177285 | 2.86299E-22 | 2.16565E-20 |
| ZRC1 | 1.047681442 | 1.03238E-06 | 1.71721E-05 |
| SNU13 | 1.195400142 | 4.37664E-05 | 0.000493946 |
| FRE3 | 1.153647335 | 5.08089E-10 | 1.41596E-08 |
| ADE3 | 1.13221044 | 2.80213E-06 | 4.09304E-05 |
| KIN82 | 1.278952486 | 5.87925E-10 | 1.58965E-08 |
| GSP1 | 1.022127262 | 0.000222718 | 0.002013017 |
| YAR1 | 1.453508427 | 2.99567E-07 | 5.45401E-06 |
| OYE3 | 1.477120456 | 1.02204E-11 | 3.47275E-10 |
| ARO1 | 1.368004705 | 5.83491E-07 | 1.02417E-05 |
| RGM1 | 1.26207715 | 5.59627E-10 | 1.53934E-08 |
| GAC1 | 1.493832249 | 4.06118E-14 | 1.80453E-12 |
| PXR1 | 1.30235573 | 2.11015E-11 | 6.9471E-10 |
| BUD27 | 1.298935286 | 3.88857E-07 | 6.921E-06 |
| VMA2 | 1.067646468 | 2.95543E-05 | 0.000347755 |
| KSP1 | 1.048173315 | 8.32619E-05 | 0.000868712 |
| ILV2 | 1.787795887 | 1.72874E-10 | 5.13292E-09 |
| MIT1 | 1.574936982 | 5.73547E-18 | 3.47078E-16 |
| GIS4 | 1.261440253 | 1.23489E-07 | 2.34925E-06 |
| AVT4 | 1.107692867 | 1.00744E-05 | 0.000132806 |
| AVT6 | 1.206525517 | 1.69234E-05 | 0.00021027 |
| MSH3 | 1.313227856 | 4.38806E-09 | 1.06014E-07 |
| NOP16 | 1.387416032 | 1.34612E-09 | 3.47694E-08 |
| PMP2 | 1.187752565 | 0.000192814 | 0.001780729 |
| YFH7 | 1.040510839 | 0.000405729 | 0.003428196 |
| UGA4 | 1.604647041 | 8.84336E-10 | 2.32193E-08 |
| SUC2 | 1.506267641 | 2.84815E-08 | 5.99244E-07 |
| DAL2 | 1.372682001 | 9.97602E-09 | 2.26384E-07 |
| KTI12 | 1.35488162 | 1.236E-06 | 1.99836E-05 |
| BNA6 | 1.200540926 | 3.1474E- 07 | 5.68141E-06 |
| NUP100 | 1.745531022 | 1.74648E-10 | 5.16145E-09 |
| NOP19 | 1.347422541 | 3.47294E-06 | 5.00387E-05 |
| PAB1 | 1.143399564 | 6.30959E-06 | 8.58482E-05 |
| YAP6 | 1.754624556 | 5.30459E-12 | 1.89356E-10 |
| UBX3 | 1.121419965 | 1.09226E-06 | 1.79799E-05 |
| PER33 | 1.433260147 | 4.50507E-06 | 6.29125E-05 |
| STE24 | 1.379720533 | 6.77396E-10 | 1.81611E-08 |
| SPO75 | 1.41546421 | 4.55372E-08 | 9.36386E-07 |
| MRT4 | 2.346979433 | 1.19269E-08 | 2.64976E-07 |
| RKM5 | 1.071321207 | 0.000140029 | 0.001356315 |
| FRE1 | 1.438547473 | 4.06837E-06 | 5.74454E-05 |
| COQ6 | 1.080486274 | 6.09252E-05 | 0.000668599 |
| ARO4 | 1.536576434 | 0.000131288 | 0.001287354 |
| GUS1 | 1.034993292 | 0.000515287 | 0.004197605 |
| ARO80 | 1.062394328 | 0.000693478 | 0.005501071 |
| ERR1 | 1.173738057 | 1.36431E-06 | 2.17264E-05 |
| ERR2 | 1.173738057 | 1.36431E-06 | 2.17264E-05 |
| MAL11 | 1.315537506 | 3.56821E-06 | 5.11792E-05 |
| FAR11 | 1.017499895 | 0.003215557 | 0.019913885 |
| GZF3 | 1.388280309 | 1.42927E-06 | 2.24791E-05 |
| CMR3 | 2.460321846 | 9.64098E-14 | 4.05687E-12 |
| SYP1 | 1.330455072 | 1.76233E-06 | 2.72454E-05 |
| ILV3 | 1.197357959 | 0.002701664 | 0.017349931 |
| APA1 | 1.304202473 | 0.000350622 | 0.003010609 |
| IML2 | 1.135591154 | 0.00264674 | 0.01705617 |
| COS10 | 1.637676185 | 8.546E-06 | 0.000113128 |
| HOL1 | 1.066441132 | 0.001095533 | 0.00806607 |
| YPQ2 | 1.005105297 | 0.005778533 | 0.031955437 |
| PLC1 | 1.255360718 | 4.42924E-06 | 6.19898E-05 |
| SOL1 | 1.051191216 | 3.98913E-05 | 0.000451817 |
| VMA6 | 1.192932877 | 1.7985E-05 | 0.000221467 |
| RPL33A | 1.36078849 | 0.00121483 | 0.008758464 |
| ARO8 | 1.364444551 | 3.30212E-05 | 0.000382877 |
| KAP95 | 1.37215159 | 6.85549E-05 | 0.000738302 |
| TIP1 | 1.09847651 | 0.000199006 | 0.001827288 |
| RRT13 | 1.044931214 | 0.000484246 | 0.004001169 |
| ARG81 | 1.700302224 | 8.21082E-05 | 0.000859498 |
| DAL3 | 1.165541421 | 0.000438638 | 0.003662451 |
| ABZ1 | 1.164055084 | 0.000102692 | 0.001042345 |
| MKK1 | 1.653611577 | 5.22281E-08 | 1.06025E-06 |
| SNT309 | 1.547509925 | 9.83937E-08 | 1.90028E-06 |
| SIZ1 | 1.065747542 | 0.002110132 | 0.013995591 |
| SEC15 | 1.090292312 | 0.000343432 | 0.002960881 |
| QNS1 | 1.294625652 | 5.32319E-05 | 0.000591321 |
| AOS1 | 1.182206751 | 0.000808268 | 0.006270738 |
| CIT3 | 1.457322169 | 5.62709E-06 | 7.77273E-05 |
| FDC1 | 1.227017655 | 0.001606312 | 0.011166854 |
| PTR2 | 1.529217751 | 8.03916E-06 | 0.000106864 |
| CHC1 | 1.466886589 | 3.18931E-05 | 0.000371152 |
| RPC82 | 1.095581179 | 0.001030924 | 0.007661391 |
| DYN1 | 1.012201794 | 0.003829259 | 0.023062663 |
| BI3 | 1.393383108 | 0.000496982 | 0.004082613 |
| DAN4 | 1.273044771 | 0.000225319 | 0.002030748 |
| DED1 | 1.389817328 | 0.000174704 | 0.001622904 |
| PDP3 | 1.174180175 | 0.005349132 | 0.029945716 |
| NSA2 | 1.725367765 | 0.001292743 | 0.009250099 |
| NUP57 | 1.042171247 | 0.005576597 | 0.031078512 |
| APQ13 | 1.339125424 | 0.002660982 | 0.017126777 |
| IRE1 | 1.193670147 | 0.002663087 | 0.017126777 |
| OAC1 | 1.45731305 | 0.009988961 | 0.049741268 |
| CAN1 | 1.417674266 | 0.001508491 | 0.010556115 |
| GIP1 | 1.25146408 | 0.000215244 | 0.001959403 |
| RRP12 | 1.379731454 | 0.00298219 | 0.018869928 |
| RRB1 | 1.495099945 | 0.003964195 | 0.023673399 |
| DAL5 | 1.242407747 | 0.001022029 | 0.007606339 |
| VAR1 | 1.691427 | 0.000285511 | 0.002509177 |
| PPX1 | 1.329682598 | 0.002642836 | 0.017048306 |
| POP3 | 1.581887054 | 0.005423444 | 0.030281693 |
| VTH1 | 2.919092817 | 4.89536E-05 | 0.000548591 |
| OPT2 | 1.167032729 | 0.008986628 | 0.046385896 |
| GRX4 | 1.459288808 | 0.000935294 | 0.007066416 |
| MMM1 | 1.311764447 | 0.004425362 | 0.025892036 |
| BSC5 | 1.668584387 | 0.006602212 | 0.03585509 |
| LEU9 | 1.520453839 | 0.004915174 | 0.028085443 |

TABLE 3

Positively enriched genes through GSEA involved in ATP metabolic processes

| PROBE | RANK IN GENE LIST | RANK METRIC SCORE | RUNNING ES | CORE ENRICHMENT |
|---|---|---|---|---|
| ATP16 | 63 | 8.436654 | 0.03682 | Yes |
| ATP4 | 65 | 8.406623 | 0.085863 | Yes |
| CYT1 | 66 | 8.342733 | 0.134733 | Yes |
| COX1 | 71 | 8.231789 | 0.182153 | Yes |
| NDI1 | 92 | 7.410284 | 0.22156 | Yes |
| ATP2 | 95 | 7.329973 | 0.264097 | Yes Yes |
| QCR2 | 96 | 7.300665 | 0.306863 | Yes |
| ATP1 | 112 | 6.827048 | 0.343854 | Yes |
| QCR7 | 114 | 6.762463 | 0.383266 | Yes |
| ATP14 | 143 | 6.156174 | 0.413728 | Yes |
| COX6 | 164 | 5.734332 | 0.443318 | Yes |
| QCR6 | 171 | 5.656575 | 0.475252 | Yes |
| ATP3 | 180 | 5.513893 | 0.505951 | Yes |
| ATP7 | 181 | 5.500307 | 0.538171 | Yes |
| ATP18 | 208 | 5.145989 | 0.563115 | Yes |
| QCR10 | 351 | 3.92371 | 0.557699 | Yes |
| ATP6 | 358 | 3.871836 | 0.579179 | Yes |
| COX9 | 387 | 3.676723 | 0.595116 | Yes |
| COX4 | 393 | 3.64507 | 0.615468 | Yes |
| QCR8 | 446 | 3.316365 | 0.624494 | Yes |
| ATP17 | 541 | 2.835934 | 0.622307 | Yes |
| OLI1 | 564 | 2.754525 | 0.634042 | Yes |
| TIM11 | 578 | 2.680261 | 0.647142 | Yes |
| COX8 | 587 | 2.641923 | 0.661018 | Yes |
| ATP5 | 596 | 2.609273 | 0.674702 | Yes |
| COX5A | 660 | 2.416687 | 0.676259 | Yes |
| TAZ1 | 661 | 2.405348 | 0.690348 | Yes |
| ATP15 | 672 | 2.358564 | 0.702164 | Yes |
| COX7 | 812 | 2.030624 | 0.686259 | No |
| RIP1 | 928 | 1.790093 | 0.673745 | No |
| COB | 1030 | 1.612267 | 0.662989 | No |
| SDH4 | 1100 | 1.506127 | 0.658012 | No |
| SDH2 | 1157 | 1.416422 | 0.655109 | No |
| ATP19 | 1237 | 1.291178 | 0.646872 | No |
| SDH1 | 1412 | 1.077623 | 0.618385 | No |
| ATP20 | 1493 | 0.981492 | 0.608134 | No |
| QCR9 | 1628 | 0.794693 | 0.585989 | No |
| COX2 | 1641 | 0.775791 | 0.588133 | No |
| GSM1 | 1907 | 0.505049 | 0.538092 | No |
| SDH3 | 2038 | 0.381977 | 0.514329 | No |
| COX3 | 2465 | 0.027086 | 0.429288 | No |
| COX5B | 2543 | -0.03951 | 0.414119 | No |
| CYC7 | 2633 | -0.13394 | 0.397104 | No |
| ATP8 | 2685 | -0.17228 | 0.387913 | No |
| CYC1 | 4439 | -2.09115 | 0.049563 | No |
| SHH4 | 5032 | -12.1954 | 0.0026 | No |

TABLE 4

Positively enriched genes through GSEA involved in the generation of metabolite and metabolite precursors

| PROBE | RANK IN GENE LIST | RANK METRIC SCORE | RUNNING ES | CORE ENRICHMENT |
|---|---|---|---|---|
| HXK1 | 18 | 14.20967 | 0.027483 | Yes |
| TDH3 | 30 | 11.90459 | 0.051341 | Yes |
| HAP4 | 43 | 10.16136 | 0.071172 | Yes |
| TDH1 | 45 | 10.10294 | 0.093123 | Yes |
| AI1 | 50 | 9.533964 | 0.113212 | Yes |
| CYT1 | 66 | 8.342733 | 0.128442 | Yes |
| ENO1 | 67 | 8.33451 | 0.146719 | Yes |
| RGI1 | 69 | 8.260607 | 0.16463 | Yes |
| COX1 | 71 | 8.231789 | 0.182477 | Yes |
| ISF1 | 79 | 7.983743 | 0.198554 | Yes |
| COR1 | 85 | 7.61409 | 0.21423 | Yes |
| GAC1 | 86 | 7.55902 | 0.230806 | Yes |
| NDI1 | 92 | 7.410284 | 0.246035 | Yes |
| QCR2 | 96 | 7.300665 | 0.261431 | Yes |
| QCR7 | 114 | 6.762463 | 0.272787 | Yes |
| GLK1 | 117 | 6.670315 | 0.287006 | Yes |
| MDH1 | 129 | 6.359677 | 0.298705 | Yes |
| COX6 | 164 | 5.734332 | 0.304332 | Yes |
| IDH1 | 170 | 5.658953 | 0.31572 | Yes |
| QCR6 | 171 | 5.656575 | 0.328125 | Yes |
| GLC7 | 172 | 5.64327 | 0.3405 | Yes |
| PET10 | 193 | 5.338264 | 0.34812 | Yes |
| TAR1 | 194 | 5.333929 | 0.359817 | Yes |
| PAH1 | 197 | 5.303865 | 0.371039 | Yes |
| FUM1 | 204 | 5.234492 | 0.381292 | Yes |
| ADH3 | 219 | 4.989831 | 0.389373 | Yes |
| KGD1 | 220 | 4.98782 | 0.400311 | Yes |
| CIT1 | 243 | 4.777311 | 0.406292 | Yes |
| GPH1 | 251 | 4.712177 | 0.415195 | Yes |
| CIT3 | 269 | 4.539937 | 0.421677 | Yes |
| PSK1 | 280 | 4.469924 | 0.429436 | Yes |
| GLC8 | 282 | 4.45793 | 0.439008 | Yes |
| IDH2 | 304 | 4.20742 | 0.443943 | Yes |
| ADE16 | 307 | 4.182589 | 0.452707 | Yes |
| QCR10 | 351 | 3.92371 | 0.452525 | Yes |
| REG1 | 369 | 3.803018 | 0.457391 | Yes |
| GDB1 | 379 | 3.72335 | 0.463717 | Yes |
| COX9 | 387 | 3.676723 | 0.470349 | Yes |
| PFK1 | 388 | 3.676702 | 0.478412 | Yes |
| COX4 | 393 | 3.64507 | 0.485588 | Yes |
| GSY2 | 410 | 3.517181 | 0.490032 | Yes |
| AAC1 | 425 | 3.42126 | 0.494674 | Yes |
| QCR8 | 446 | 3.316365 | 0.497859 | Yes |
| GDS1 | 455 | 3.256814 | 0.503367 | Yes |
| ADH5 | 505 | 2.963919 | 0.499854 | Yes |
| BMH2 | 509 | 2.961417 | 0.505735 | Yes |
| COX13 | 520 | 2.917452 | 0.51009 | Yes |
| GIP2 | 536 | 2.859262 | 0.513295 | Yes |
| PCL10 | 550 | 2.796241 | 0.51677 | Yes |
| GCR2 | 567 | 2.743029 | 0.519516 | Yes |
| GSY1 | 568 | 2.740006 | 0.525525 | Yes |
| ATF1 | 571 | 2.724546 | 0.531019 | Yes |
| COX8 | 587 | 2.641923 | 0.53382 | Yes |
| GLG1 | 640 | 2.473786 | 0.528619 | Yes |
| COX5A | 660 | 2.416687 | 0.530036 | Yes |
| TAZ1 | 661 | 2.405348 | 0.535311 | Yes |
| GLC3 | 663 | 2.40101 | 0.540372 | Yes |
| PFK27 | 665 | 2.39152 | 0.545412 | Yes |
| CSF1 | 740 | 2.199048 | 0.535114 | No |
| NDE1 | 754 | 2.148085 | 0.537168 | No |
| PUF3 | 761 | 2.139955 | 0.540635 | No |
| COX7 | 812 | 2.030624 | 0.534871 | No |
| ADH1 | 862 | 1.908086 | 0.529043 | No |
| ADH4 | 876 | 1.870776 | 0.530489 | No |
| RIP1 | 928 | 1.790093 | 0.523994 | No |
| DLD1 | 1021 | 1.626894 | 0.508763 | No |
| COB | 1030 | 1.612267 | 0.510664 | No |
| PPG1 | 1050 | 1.584502 | 0.510256 | No |
| IGD1 | 1095 | 1.51167 | 0.504581 | No |
| SDH4 | 1100 | 1.506127 | 0.507066 | No |
| LSC2 | 1119 | 1.4488 | 0.506622 | No |
| TYE7 | 1122 | 1.473071 | 0.509443 | No |
| MBR1 | 1142 | 1.440608 | 0.50872 | No |
| SDH2 | 1157 | 1.416422 | 0.508966 | No |
| PGK1 | 1193 | 1.365402 | 0.504808 | No |
| HAP1 | 1195 | 1.360008 | 0.507586 | No |
| ETR1 | 1294 | 1.216719 | 0.49023 | No |
| SDH1 | 1412 | 1.077623 | 0.468686 | No |
| RIB3 | 1448 | 1.032143 | 0.463798 | No |
| PCL6 | 1477 | 1.002301 | 0.460275 | No |
| RAP1 | 1490 | 0.983831 | 0.45998 | No |
| PFK2 | 1491 | 0.983416 | 0.462137 | No |
| SHP1 | 1511 | 0.954816 | 0.460348 | No |
| YMR31 | 1513 | 0.952786 | 0.462233 | No |
| PCL7 | 1534 | 0.925702 | 0.460177 | No |
| PDC5 | 1563 | 0.881775 | 0.456389 | No |
| QCR9 | 1628 | 0.794693 | 0.445055 | No |
| CDC19 | 1640 | 0.778471 | 0.444514 | No |
| COX2 | 1641 | 0.775791 | 0.446215 | No |
| NCA2 | 1654 | 0.765868 | 0.445443 | No |
| GSM1 | 1907 | 0.50549 | 0.395059 | No |
| SDH3 | 2038 | 0.381977 | 0.369333 | No |
| ALG6 | 2088 | 0.32789 | 0.36004 | No |
| PDC1 | 2100 | 0.317477 | 0.358489 | No |
| HAP2 | 2103 | 0.316796 | 0.358775 | No |
| AAC3 | 2140 | 0.294268 | 0.352064 | No |
| PGI1 | 2217 | 0.227661 | 0.337034 | No |
| MCT1 | 2277 | 0.16628 | 0.325343 | No |
| GCR1 | 2299 | 0.144194 | 0.321368 | No |
| UGP1 | 2377 | 0.082331 | 0.305815 | No |
| COQ10 | 2403 | 0.060705 | 0.30084 | No |
| COX3 | 2465 | 0.027086 | 0.288435 | No |
| COX5B | 2543 | -0.03951 | 0.272788 | No |
| YPI1 | 2561 | -0.06379 | 0.269455 | No |
| PSK2 | 2616 | -0.11901 | 0.258682 | No |
| CYC7 | 2633 | -0.13394 | 0.255706 | No |
| PDC2 | 2694 | -0.18021 | 0.243841 | No |
| COQ5 | 2744 | -0.22164 | 0.234315 | No |
| FBA1 | 2764 | -0.23768 | 0.230954 | No |
| PCL8 | 2806 | 0.26912 | 0.223167 | No |
| PGM2 | 2900 | -0.34928 | 0.20493 | No |
| JAC1 | 2984 | -0.42152 | 0.188894 | No |
| LSC1 | 3061 | -0.4711 | 0.174398 | No |
| SGA1 | 3082 | -0.48928 | 0.171385 | No |
| PPA2 | 3225 | -0.62629 | 0.143743 | No |
| CBP1 | 3240 | -0.64405 | 0.142295 | No |
| GPM1 | 3295 | -0.69713 | 0.13279 | No |
| AAP1 | 3425 | -0.81869 | 0.108226 | No |
| OAR1 | 3433 | -0.82541 | 0.108606 | No |
| HXK2 | 3666 | -1.02541 | 0.063449 | No |
| PET20 | 3700 | -1.06073 | 0.059033 | No |
| HAP3 | 3818 | -1.17041 | 0.037692 | No |
| PHO85 | 3910 | -1.28298 | 0.021912 | No |
| PIG1 | 3954 | -1.33801 | 0.016059 | No |
| TPI1 | 3956 | -1.33879 | 0.018791 | No |
| COX20 | 3971 | -1.36016 | 0.018913 | No |
| MIX14 | 4007 | -1.40728 | 0.014847 | No |
| MIX17 | 4018 | -1.42181 | 0.015922 | No |
| SLS1 | 4110 | -1.53682 | 6.98E-04 | No |
| ATF2 | 4114 | -1.5412 | 0.003465 | No |
| SDH5 | 4126 | -1.56529 | 0.00465 | No |
| ALG7 | 4280 | -1.80189 | -0.02266 | No |
| PIG2 | 4293 | -1.8148 | -0.02113 | No |
| CYC1 | 4439 | -2.09115 | -0.04618 | No |
| ENO2 | 4447 | -2.11443 | -0.04297 | No |
| PET9 | 4548 | -2.36255 | -0.05822 | No |
| RMD9 | 4549 | -2.36316 | -0.05304 | No |
| MNP1 | 4563 | -2.40112 | -0.05043 | No |
| HAP5 | 4588 | -2.45789 | -0.04994 | No |
| ACO1 | 4640 | -2.58321 | -0.0547 | No |
| TDH2 | 4709 | -2.83234 | -0.06238 | No |
| GLG2 | 4717 | -2.85237 | -0.05756 | No |
| RSF1 | 4722 | -2.86983 | -0.05208 | No |
| PGM1 | 4743 | -2.97564 | -0.04965 | No |
| COX11 | 4823 | -3.40976 | -0.05831 | No |
| NDE2 | 4878 | -3.81771 | -0.06097 | No |
| SOD1 | 4886 | -3.87253 | -0.05391 | No |
| ACS1 | 4929 | -4.40536 | -0.05283 | No |
| MAM33 | 4944 | -4.66876 | -0.04545 | No |
| BMH1 | 4967 | -5.13864 | -0.03868 | No |
| SHH4 | 5032 | -12.1954 | -0.02501 | No |
| RGI2 | 5034 | -12.5252 | 0.002248 | No |

TABLE 5

Cell size measured via a Coulter counter

| Strain | Mean Diameter (µm) | Median | Standard Deviation | Count |
|---|---|---|---|---|
| W303α | 5.747 | 5.688 | 1.7 | 208.181 |
| W303α | 5.79 | 5.708 | 1.639 | 180.954 |
| W303α + light | 5.797 | 5.719 | 1.657 | 189.964 |
| W303α + Cd + light | 5.815 | 5.734 | 1.689 | 201.924 |
| ΔMet17 | 5.453 | 5.419 | 1.609 | 126.784 |
| ΔMet17 + Cd | 5.574 | 5.549 | 1.652 | 141.717 |
| ΔMet17 + light | 5.563 | 5.543 | 1.649 | 144.139 |
| ΔMet17 + Cd + light | 5.55 | 5.516 | 1.687 | 178.268 |
| Y567 | 5.669 | 5.583 | 1.744 | 229.242 |
| Y567 + Cd | 5.765 | 5.676 | 1.683 | 205.561 |
| Y567 + light | 5.807 | 5.746 | 1.596 | 172.302 |
| Y567 + Cd + light | 5.81 | 5.739 | 1.683 | 201.655 |
| Y567::ΔMet17 | 5.533 | 5.495 | 1.648 | 143.732 |
| Y567::ΔMet17 + Cd | 5.593 | 5.561 | 1.667 | 163.638 |
| Y567::ΔMet17 + light | 5.597 | 5.561 | 1.666 | 163.638 |
| Y567::ΔMet17 + Cd + light | 5.603 | 5.573 | 1.656 | 141.52 |

TABLE 6

Doubling times from growth experiments in synthetic media

| Strain Mean | Doubling Time (minutes) | Standard Deviation (minutes) |
|---|---|---|
| W303α | 1.41 | 4.26 |
| W303α | 140 | 3.08 |
| W303α + light | 143 | 5.25 |
| W303α + Cd + light | 142 | 3.71 |
| ΔMet17 | 144 | 3.44 |
| ΔMet17 + Cd | 140 | 3.82 |
| ΔMet17 + light | 145 | 5.14 |
| ΔMet17 + Cd + light | 142 | 4.71 |
| Y567 | 137 | 3.48 |
| Y567 + Cd | 142 | 3.36 |
| Y567 + light | 143 | 4.37 |
| Y567 + Cd + light | 140 | 3.32 |
| Y567::ΔMet17 | 140 | 3.56 |
| Y567::ΔMet17 + Cd | 141 | 4.08 |
| Y567::ΔMet17 + light | 142 | 5.16 |
| Y567::ΔMet17 + Cd + light | 141 | 4.44 |

Other embodiments are within the scope of the following claims.

What is claimed is:

1. A system for production of a chemical product comprising:
a cell;
a nanoparticle on a surface of the cell; and
an irradiation unit configured to expose the cell to irradiation.

2. The system of claim 1, wherein the cell is a yeast cell.

3. The system of claim 1, wherein a thiol synthesis pathway is deleted from the cell.

4. The system of claim 3, wherein the thiol synthesis pathway includes Met17.

5. The system of claim 1, wherein the nanoparticle includes cadmium.

6. The system of claim 1, wherein the nanoparticle includes cadmium sulfide.

7. The system of claim 1, wherein the irradiation unit includes an ultraviolet (UV) light source.

8. The system of claim 1, wherein the system includes a bioreactor including the irradiation unit configured to irradiate contents of the bioreactor.

9. A method of producing a chemical product comprising:
providing a cell having a nanoparticle on a surface of the cell;
exposing the cell to a precursor;
irradiating the cell;
converting the precursor to a chemical product with the cell; and
collecting the chemical product.

10. The method of claim 9, wherein the cell is a yeast cell.

11. The method of claim 9, wherein a thiol synthesis pathway is deleted from the cell.

12. The method of claim 11, wherein the thiol synthesis pathway includes Met17.

13. The method of claim 9, wherein the nanoparticle includes cadmium.

14. The method of claim 9, wherein the nanoparticle includes cadmium sulfide.

15. The method of claim 9, wherein the irradiating the cell includes irradiating ultraviolet (UV) light.

16. The method of claim 9, wherein the chemical product is a biofuel.

17. The method of claim 9, wherein the chemical product is ethanol.

18. The method of claim 9, wherein the precursor includes glucose.

19. The method of claim 9, wherein the precursor includes carbon dioxide.
